(19) United States
(12) Patent Application Publication
(10) Pub. No.: US 2025/0041280 A1
(43) Pub. Date: Feb. 6, 2025

(54) USE OF HETEROCYCLIC COMPOUNDS IN ALLEVIATING ADVERSE REACTIONS CAUSED BY CHEMOTHERAPEUTIC DRUGS

(71) Applicant: TSINGHUA UNIVERSITY, Beijing (CN)

(72) Inventors: Heng LI, Beijing (CN); Wei WU, Beijing (CN); Qing XUE, Beijing (CN)

(73) Assignee: TSINGHUA UNIVERSITY, Beijing (CN)

(21) Appl. No.: 18/719,504

(22) PCT Filed: Dec. 13, 2022
(86) PCT No.: PCT/CN2022/138506

§ 371 Date: Jun. 13, 2024

(30) Foreign Application Priority Data
Dec. 13, 2021 (CN) 202111520310.2

(51) Int. Cl. A61K 31/422

(52) U.S. Cl. CPC A61K 31/422

(57) ABSTRACT

The use of a heterocyclic compound in reducing adverse reactions caused by chemotherapy drugs. The present invention specifically relates to use of a compound as represented by formula (1) or a pharmaceutically acceptable form thereof in the preparation of a drug for reducing adverse reactions caused by chemotherapy drugs.

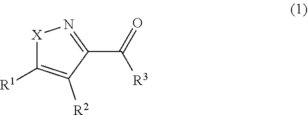

### CROSS REFERENCES TO RELATED APPLICATIONS

This application claims the benefit and priority of Chinese Patent Application CN202111520310.2 filed on Dec. 16, 2021, the content of which is incorporated herein by reference in its entirety.

### TECHNICAL FIELD

This application belongs to the field of medicaments, and relates to use of heterocyclic compounds in the preparation of a medicament for alleviating adverse reactions caused by chemotherapeutic drugs.

### BACKGROUND ART

The most basic and broad-spectrum methods in cancer treatment are radiotherapy and chemotherapy, also referred to as chemoradiotherapy. Both will damage the normal cells in a patient's body, especially the cells in relatively fast-renewing tissues, thereby causing various adverse reactions (so-called side effects) of radiotherapy and chemotherapy, and seriously affecting the life quality of cancer patients. Some patients have low tolerance to adverse reactions caused by radiotherapy and chemotherapy, thus the dose of radiotherapy and chemotherapy has to be reduced or the treatment cycle has to be adjusted, thereby greatly affecting the treatment effect of cancer.

The intestinal tract is the organ with the fastest cell renewal rate in the human body, and a large number of rapidly proliferating stem cells and precursor cells are sensitive to radiotherapy and chemotherapy, so intestinal damage is one of the main side effects of radiotherapy and chemotherapy. Relevant studies have shown that, about 40% to 60% of cancer patients who receive chemotherapy or radiation therapy will be accompanied by adverse reactions in the digestive tract, and among patients who receive pre-chemotherapy before stem cell transplantation, this proportion will almost reach 100%. However, so far, there is no approved adjuvant drug that can specifically alleviate intestinal adverse reactions in chemotherapy and radiotherapy. Therefore, it is of great significance to find new drugs to alleviate the adverse reactions caused by radiotherapy and chemotherapy.

### SUMMARY

Through a lot of experiments, the inventors of the present application unexpectedly found that the heterocyclic compound represented by formula (1) or a pharmaceutically acceptable form thereof has the utility of alleviating adverse reactions caused by chemotherapy drugs, thereby completing the present application. In a first aspect, the present application provides a compound represented by formula (1) or a pharmaceutically acceptable form thereof in the preparation of a medicament for alleviating adverse reactions caused by chemotherapy drugs, the structure of the compound represented by formula (1) is as follows:

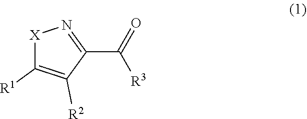

wherein:
R¹ is selected from C₁₋₆ alkyl, C₃₋₆ cycloalkyl, 3-8 membered heterocycloalkyl, C₆₋₁₀ aryl or 5-10 membered heteroaryl; and wherein R¹ is optionally substituted by one or more R^{a};
the R^{a} is selected from hydrogen, hydroxyl, halogen, nitro, cyano, C₆₋₁₀ aryl, 5-10 membered heteroaryl, C₁₋₆ alkyl, —OC₁₋₆ alkyl, C₂₋₆ alkenyl, —OC₂₋₆ alkenyl, C₂₋₆ alkynyl, —OC₂₋₆ alkynyl, C₃₋₆ cycloalkyl, —C₁₋₆ alkylene-C₃₋₆ cycloalkyl, —OC₃₋₆ cycloalkyl, —OC₁₋₆ alkylene-C₃₋₆ cycloalkyl, —C₁₋₆ alkylene-C₆₋₁₀ aryl, —OC₆₋₁₀ aryl, —OC₁₋₆ alkylene-C₆₋₁₀ aryl, CHO, —(CO)R^{b}, —O(CO)R^{b}, —O(CO)OR^{b}, —C₁₋₆ alkylene-OR^{b}, —OC₂₋₆ alkylene-OR^{b}, —C₁₋₆ alkylene-(CO)R^{b}, —OC₁₋₆ alkylene-(CO)R^{b}, —CO₂R^{b}, —C₁₋₆ alkylene-CO₂R^{b}, —OC₁₋₆ alkylene-CO₂R^{b}, —NR^{b}R^{c}, —C₁₋₆ alkylene-NR^{b}R^{c}, —OC₂₋₆ alkylene-NR^{b}R^{c}, —C₁₋₆ alkylene-(CO)NR^{b}R^{c}, —OC₁₋₆ alkylene-(CO)NR^{b}R^{c}, —NR^{b}(CO)R^{c}, —C₁₋₆ alkylene-NR^{b}(CO)R^{c}, —OC₂₋₆ alkylene-NR^{b}(CO)R^{c}, —NR^{b}(CO)NR^{b}R^{c}, —C₁₋₆ alkylene-NR^{b}(CO)NR^{b}R^{c}, —SR^{b}, —C₁₋₆ alkylene-SR^{b}, —OC₂₋₆ alkylene-SR^{b}, —(SO)R^{b}, —C₁₋₆ alkylene-(SO)R^{b}, —OC₂₋₆ alkylene-(SO)R^{b}, —SO₂R^{b}, —C₁₋₆ alkylene-SO₂R^{b}, —OC₂₋₆ alkylene-SO₂R^{b}, —(SO₂)NR^{b}R^{c}, —C₁₋₆ alkylene-(SO₂)NR^{b}R^{c}, —OC₁₋₆ alkylene-(SO₂)NR^{b}W, —NR^{b}(SO₂)R^{c}, —C₁₋₆ alkylene-NR^{b}(SO₂)R^{c}, —OC₂₋₆ alkylene-NR^{b}(SO₂)R^{c}, —NR^{b}(SO₂)NR^{b}W, —C₁₋₆ alkylene-NR^{b}(SO₂)NR^{b}R^{c}, —OC₂₋₆ alkylene-NR^{b}(SO₂)NR^{b}W, —(CO)NR^{b}R^{c}, —O(CO)NR^{b}R^{c}, —NR^{b}OR^{c}, —NR^{b}(CO)OR^{c}, —C₁₋₆ alkylene-NR^{b}(CO)OR^{c} or —OC₂₋₆ alkylene-NR^{b}(CO)OR^{c}, and wherein the aryl, heteroaryl, alkyl, alkenyl, alkynyl, cycloalkyl, alkylene are optionally substituted by one or more substituents selected from the group consisting of: halogen, hydroxyl, cyano, nitro, C₁₋₆ alkyl, —OC₁₋₆ alkyl and C₃₋₆ cycloalkyl;
R² is selected from hydrogen, hydroxyl, halogen, nitro, cyano, C₆₋₁₀ aryl, 5-10 membered heteroaryl, C₁₋₆ alkyl, —OC₁₋₆ alkyl, C₂₋₆ alkenyl, —OC₂₋₆ alkenyl, C₂₋₆ alkynyl, —OC₂₋₆ alkynyl, C₃₋₆ cycloalkyl, —C₁₋₆ alkylene-C₃₋₆ cycloalkyl, —OC₃₋₆ cycloalkyl, —OC₁₋₆ alkylene-C₃₋₆ cycloalkyl, —C₁₋₆ alkylene-C₆₋₁₀ aryl, —OC₆₋₁₀ aryl, —OC₁₋₆ alkylene-C₆₋₁₀ aryl, CHO, —(CO)R^{b}, —O(CO)R^{b}, —O(CO)OR^{b}, —C₁₋₆ alkylene-OR^{b}, —OC₂₋₆ alkylene-OR^{b}, —C₁₋₆ alkylene-(CO)R^{b}, —OC₁₋₆ alkylene-(CO)R^{b}, —CO₂R^{b}, —C₁₋₆ alkylene-CO₂R^{b}, —OC₁₋₆ alkylene-CO₂R^{b}, —NR^{b}R^{c}, —C₁₋₆ alkylene-NR^{b}R^{c}, —OC₂₋₆ alkylene-NR^{b}R^{c}, —C₁₋₆ alkylene-(CO)NR^{b}R^{c}, —OC₁₋₆ alkylene-(CO)NR^{b}R^{c}, —NR^{b}(CO)R^{c}, —C₁₋₆ alkylene-NR^{b}(CO)R^{c}, —OC₂₋₆ alkylene-NR^{b}(CO)R^{c}, —NR^{b}(CO)NR^{b}R^{c}, —C₁₋₆ alkylene-NR^{b}(CO)NR^{b}R^{c}, —SR^{b}, —C₁₋₆ alkylene-SR^{b}, —OC₂₋₆ alkylene-SR^{b}, —(SO)R^{b}, —C₁₋₆ alkylene-(SO)R^{b}, —OC₂₋₆ alkylene-(SO)R^{b}, —SO₂R^{b}, —C₁₋₆ alkylene-SO₂R^{b}, —OC₂₋₆ alkylene-SO₂R^{b}, —(SO₂)NR^{b}R^{c}, —C₁₋₆ alkylene-(SO₂)NR^{b}R^{c}, —OC₂₋₆ alkylene-(SO₂)NR^{b}W, —NR^{b}(SO₂)R^{c}, —C₁₋₆ alkylene-NR^{b}(SO₂)R^{c}, —OC₂₋₆ alkylene-NR^{b}(SO₂)R^{c}, —NR^{b}(SO₂)NR^{b}W, —C₁₋₆ alkylene-NR^{b}(SO₂)NR^{b}R^{c}, —OC₂₋₆ alkylene-NR^{b}(SO₂)NR^{b}W, —(CO)NR^{b}R^{c}, —O(CO)NR^{b}R^{c}, —NR^{b}OR^{c}, —NR^{b}(CO)OR^{c}, —C₁₋₆ alkylene-NR^{b}(CO)OR^{c} or —OC₂₋₆ alkylene-NR^{b}(CO)OR^{c}, and wherein the aryl, heteroaryl, alkyl, alkenyl, alkynyl, cycloalkyl, alkylene are optionally substituted by one or more substituents selected from the group consisting of: halogen, hydroxyl, cyano, nitro, C₁₋₆ alkyl, —OC₁₋₆ alkyl and C₃₋₆ cycloalkyl;
R^{b} and R^{c} are independently selected from hydrogen, C₁₋₆ alkyl, —C₁₋₆ alkylene-C₆₋₁₀ aryl, —C₁₋₆ alkylene-(5-10 membered heteroaryl), C₃₋₇ cycloalkyl or C₆₋₁₀ aryl;
R³ is selected from —OR⁷ or —NR⁷R⁸;
R⁷ and R⁸ are independently selected from hydrogen, hydroxyl, C₁₋₆ alkyl, —OC₁₋₆ alkyl, C₂₋₆ alkenyl, C₂₋₆ alkynyl, —C₁₋₆ alkylene-C₆₋₁₀ aryl, —C₁₋₆ alkylene-(5-10 membered heteroaryl), —C₁₋₆ alkylene-(3-8 membered heterocycloalkyl), C₃₋₇ cycloalkyl or C₆₋₁₀ aryl, and wherein the aryl, heteroaryl, alkyl, alkenyl, alkynyl, cycloalkyl, and alkylene are optionally substituted by one or more substituents selected from the group consisting of: halogen, hydroxyl, cyano, nitro, C₁₋₆ alkyl, —OC₁₋₆ alkyl and C₃₋₆ cycloalkyl;
alternatively, R⁷ and R⁸ form a 3-8 membered heterocycloalkyl or a 5-10 membered heteroaryl together with the N atom to which they are attached;
X is selected from 0 or NR^{d}, and R^{d} is selected from hydrogen, C₁₋₆ alkyl, —C₁₋₆ alkylene-C₆₋₁₀ aryl, —C₁₋₆ alkylene-(5-10 membered heteroaryl), C₃₋₇ cycloalkyl or C₆₋₁₀ aryl; and
the pharmaceutically acceptable form is selected from pharmaceutically acceptable salts, esters, stereoisomers, tautomers, solvates, N-oxides, isotopic labels, metabolites or prodrugs.

In some embodiments, in the compound represented by formula (1), R¹ is selected from C₁₋₆ alkyl, C₆₋₁₀ aryl or 5-10 membered heteroaryl; preferably, R1 is selected from pyridyl, methyl, phenyl, thienyl, benzothienyl, furyl, benzofuryl, pyrrolyl or thiazolyl; more preferably, R¹ is selected from

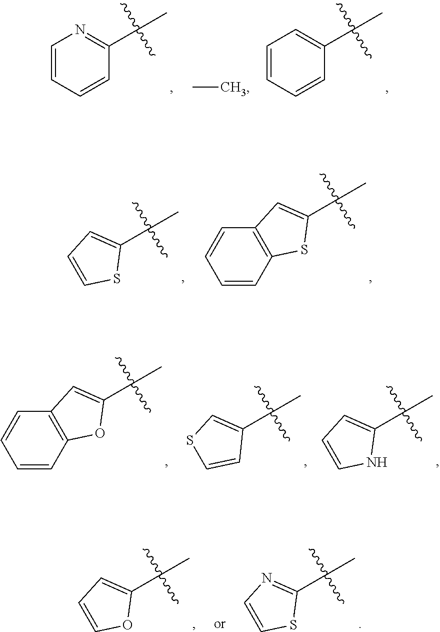

In some embodiments, the compound represented by the formula (1) is a compound represented by the following formula (2):

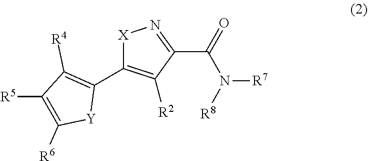

wherein:
R⁴, R⁵ and R⁶ are independently selected from hydrogen, hydroxyl, halogen, nitro, cyano, C₆₋₁₀ aryl, 5-10 membered heteroaryl, C₁₋₆ alkyl, —OC₁₋₆ alkyl, C₂₋₆ alkenyl, —OC₂₋₆ alkenyl, C₂₋₆ alkynyl, —OC₂₋₆ alkynyl, C₃₋₆ cycloalkyl, —C₁₋₆ alkylene-C₃₋₆ cycloalkyl, —OC₃₋₆ cycloalkyl, —OC₁₋₆ alkylene-C₃₋₆ cycloalkyl, —C₁₋₆ alkylene-C₆₋₁₀ aryl, —OC₆₋₁₀ aryl, —OC₁₋₆ alkylene-C₆₋₁₀ aryl, CHO, —(CO)R^{b}, —O(CO)R^{b}, —O(CO)OR^{b}, —C₁₋₆ alkylene-OR^{b}, —OC₂₋₆ alkylene-OR^{b}, —C₁₋₆ alkylene-(CO)R^{b}, —OC₁₋₆ alkylene-(CO)R^{b}, —CO₂R^{b}, —C₁₋₆ alkylene-CO₂R^{b} or —OC₁₋₆ alkylene-CO₂R^{b}, and wherein the aryl, heteroaryl, alkyl, alkenyl, alkynyl, cycloalkyl, and alkylene are optionally substituted by one or more substituents selected from the group consisting of: halogen, hydroxyl, cyano, nitro, C₁₋₆ alkyl, —OC₁₋₆ alkyl and C₃₋₆ cycloalkyl;
Y is selected from S, O or NH; and
X, R^{b}, R², R⁷ and R⁸ are as defined in formula (1).

In some embodiments, the compound represented by formula (2) is a compound represented by formula (3):

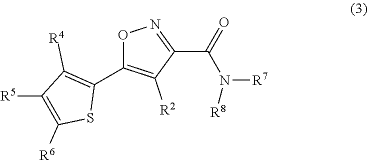

wherein R⁴, R⁵, R⁶, R², R⁷ and R⁸ are as defined in formula (2).

In some embodiments, in the compound represented by formula (2) or formula (3), R⁴, R⁵ and R⁶ are independently selected from hydrogen, hydroxyl, halogen, nitro, cyano, C₆₋₁₀ aryl, 5-10 membered heteroaryl, C₁₋₆ alkyl, —OC₁₋₆ alkyl, C₂₋₆ alkenyl, —OC₂₋₆ alkenyl, C₂₋₆ alkynyl, —OC₂₋₆ alkynyl, C₃₋₆ cycloalkyl, —OC₃₋₆ cycloalkyl or —OC₆₋₁₀ aryl, and wherein the aryl, heteroaryl, alkyl, alkenyl, alkynyl, cycloalkyl are optionally substituted by one or more substituents selected from the group consisting of: halogen, hydroxyl, cyano, nitro and C₁₋₆ alkyl;
preferably, R⁴, R⁵ and R⁶ are independently selected from hydrogen, hydroxyl, halogen, nitro, cyano, C₆₋₁₀ aryl or C₁₋₆ alkyl; more preferably, R⁴, R⁵ and R⁶ are independently selected from hydrogen, hydroxyl, halogen, nitro, cyano, phenyl, methyl or n-butyl.

In some embodiments, in the compounds represented by formula (1), formula (2) or formula (3), R² is selected from hydrogen, hydroxyl, halogen, nitro, cyano, C₆₋₁₀ aryl, 5-10 membered heteroaryl, C₁₋₆ alkyl, —OC₁₋₆ alkyl, C₂₋₆ alkenyl, —OC₂₋₆ alkenyl, C₂₋₆ alkynyl, —OC₂₋₆ alkynyl, C₃₋₆ cycloalkyl, —OC₃₋₆ cycloalkyl, —OC₆₋₁₀ aryl or CHO; preferably, R² is selected from hydrogen, hydroxyl, halogen, nitro, cyano or methyl.

In some embodiments, in the compound represented by formula (1), formula (2) or formula (3), R⁷ and R⁸ are independently selected from hydrogen, hydroxyl, C₁₋₆ alkyl, —OC₁₋₆ alkyl, —C₁₋₆ alkylene-(5-10 membered heteroaryl), —C₁₋₆ alkylene-(3-8 membered heterocycloalkyl), C₃₋₇ cycloalkyl or C₆₋₁₀ aryl, and wherein the aryl, heteroaryl, alkyl, cycloalkyl, and alkylene are optionally substituted by one or more substituents selected from the group consisting of: halogen, hydroxyl, cyano, nitro, and C₁₋₆ alkyl; or R⁷ and R⁸ form a 3-8 membered heterocycloalkyl together with the N atom to which they are attached;
preferably, R⁷ and R⁸ are independently selected from hydrogen, hydroxyl, cyclopropyl, cyclobutyl, cyclopentyl, cyclohexyl, tert-butyl,

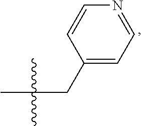 [SMILES: *Cc1ccncc1]

methyl, n-butyl,

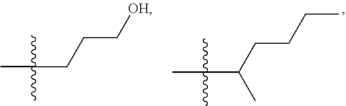

n-propyl,

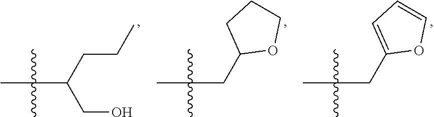

methoxy, ethoxy, or

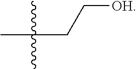 [SMILES: *CCO]

In some embodiments, the compound represented by formula (1) is selected from the following compounds:

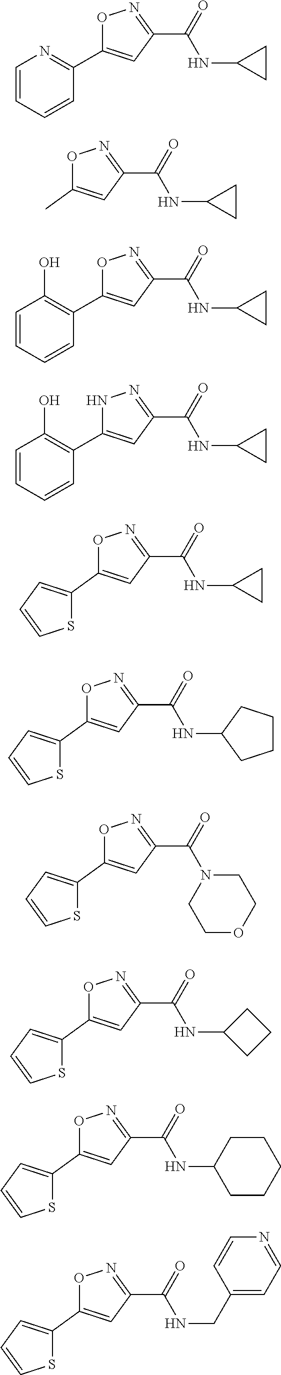

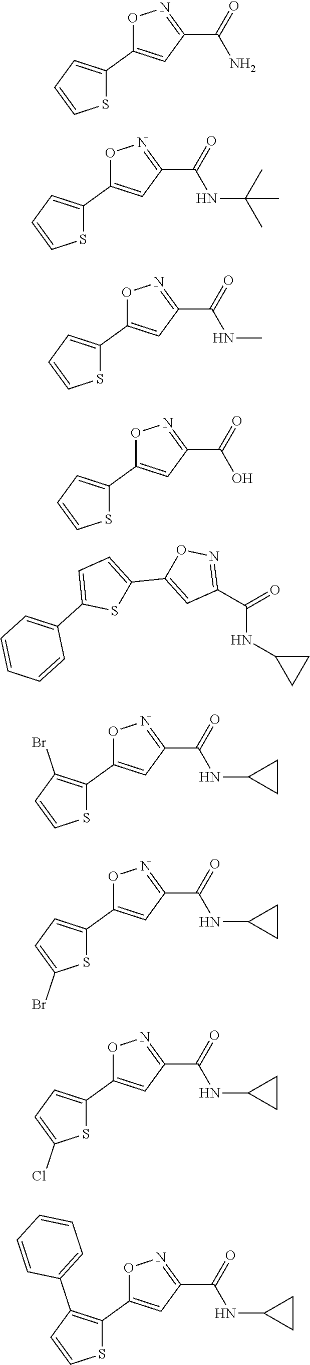

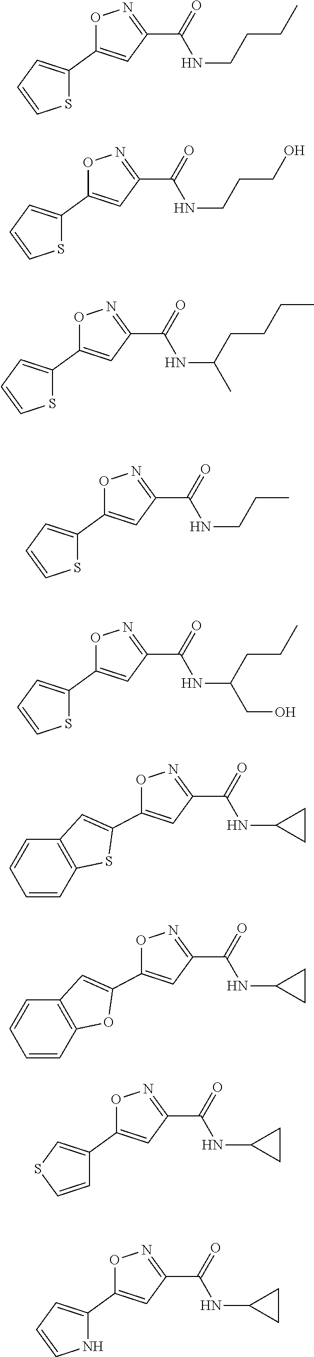

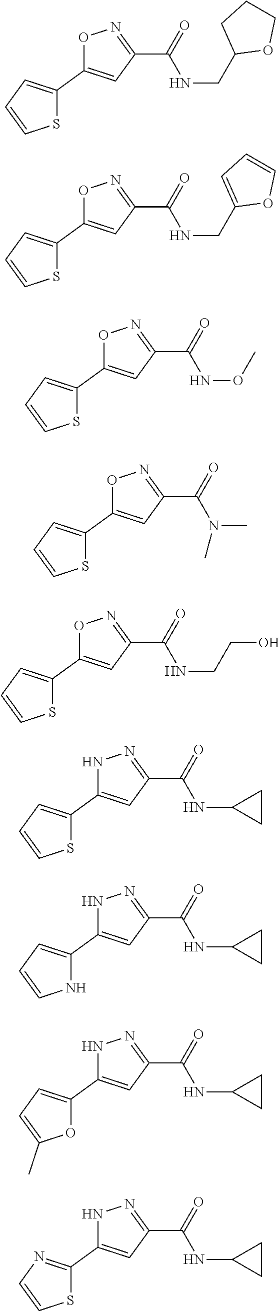

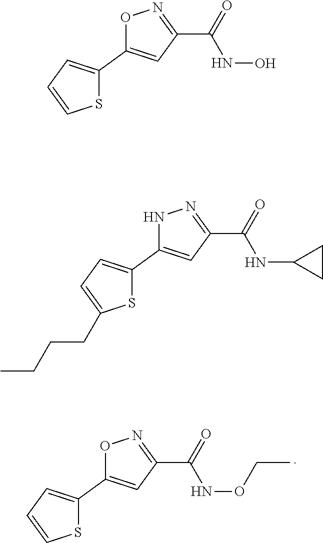 [SMILES: O=C(NO)c1cc(-c2cccs2)on1 ; CCCCc1ccc(-c2cc(C(=O)NC3CC3)n[nH]2)s1 ; CCONC(=O)c1cc(-c2cccs2)on1]

In a second aspect, the present application provides a compound represented by formula (1) to formula (3) or a pharmaceutically acceptable form thereof in the preparation of a medicament for alleviating adverse reactions caused by chemotherapy drugs, wherein the adverse reaction is an intestinal adverse reaction.

In a third aspect, the present application provides a method for alleviating adverse reactions caused by chemotherapeutic drugs, wherein the method comprises the following steps: administering an effective amount of a compound represented by the above formula (1) to formula (3) or a pharmaceutically acceptable form to an individual in need thereof.

The application is not to be limited to the particular embodiments described herein; it would be understood that the terms used herein are for the purpose of description only and are not intended to be limiting of the particular embodiments.

### General Terms and Definitions

Unless otherwise defined, the meanings of terms used herein are the same as those commonly understood by those skilled in the art. The terms used herein are intended to refer to the technology generally understood in the art, including changes or equivalent replacements of the technology obvious to those skilled in the art. Although the following terms are readily understood by those skilled in the art, they are set forth below in order to better explain the present application.

The terms “include/including”, “comprise/comprising”, “have/has/having” or “involve/involving” and other variations thereof herein refer to an inclusive or open collective concept, and do not exclude other un-recited elements or method steps. Those skilled in the art should understand that, the above terms such as “comprise/comprising” cover the meaning of “consist/consisting of”.

The term “one or more”, or a similar expression “at least one” means for example 1, 2, 3, 4, 5, 6, 7, 8, 9, 10 or more.

Where the lower and upper limits of a numerical range are disclosed, any value or any sub-range falling within that range is specifically disclosed. In particular, every numerical range (eg., in the form of “about a to b”, or equivalent “approximately a to b”, or equivalent “about a-b”) of a parameter disclosed herein is to be understood to encompass each value within such a range and its subranges. For example, “C₁₋₆” should be understood as encompassing any sub-range and every point value, such as C₂₋₅, C₃₋₄, C₁₋₂, C₁₋₃, C₁₋₄, C₁₋₅, etc., and C₁, C₂, C₃, C₄, C₅, C₆, etc. For another example, “3-10 membered” should be understood as encompassing any sub-range and every point value, such as 3-4 membered, 3-5 membered, 3-6 membered, 3-7 membered, 3-8 membered, 3-9 membered, 4-5 membered, 4-6 membered, 4-7 membered, 4-8 membered, 5-7 membered, 5-8 membered, 6-7 membered, etc., and 3-, 4-, 5-, 6-, 7-, 8-, 9-, 10 membered, etc.

As used herein, when used alone or in combination with other groups, the term “alkyl” means a linear or branched saturated aliphatic alkyl. For example, the term “C₁₋₆ alkyl” used herein means a saturated linear or branched alkyl having 1-6 carbon atoms (eg., 1, 2, 3, 4, 5 or 6 carbon atoms). For example, “C₁₋₆ alkyl” may be methyl, ethyl, n-propyl, isopropyl, n-butyl, isobutyl, sec-butyl, tert-butyl, n-pentyl, isopentyl, neopentyl base or n-hexyl, etc.

As used herein, when used alone or in combination with other groups, the term “alkylene” means a saturated linear or branched divalent alkyl. For example, the term “C₁₋₆ alkylene” used herein means a saturated linear or branched divalent alkyl having 1-6 carbon atoms, such as methylene, ethylene, propylene or butylene, etc.

As used herein, when used alone or in combination with other groups, the term “cycloalkyl” means a saturated or partially saturated, monocyclic or polycyclic (such as bicyclic, e.g., fused ring, bridging ring, or spirocyclic) non-aromatic alkyl. For example, the term “C₃₋₆ cycloalkyl” used herein means a cycloalkyl having 3 to 6 carbon atoms. For example, cycloalkyl may be cyclopropyl, cyclobutyl, cyclopentyl, cyclohexyl, or bicyclo[2.2.1]heptyl, etc.

As used herein, when used alone or in combination with other groups, the term “heterocycloalkyl” means a saturated or partially saturated, monocyclic or polycyclic (such as bicyclic, e.g., fused ring, bridging ring, or spirocyclic) non-aromatic group, the ring atoms of which are composed of carbon atoms and at least one heteroatom selected from N, O and S. The heterocyclyl may be attached to the rest of the molecule through any ring atom, provided the valence requirements are met. For example, the term “3-8 membered heterocycloalkyl” used herein refers to a heterocyclyl having 3 to 8 ring atoms. For example, the heterocyclyl may be oxiranyl, aziridinyl, azetidinyl, oxetanyl, tetrahydrofuranyl, dioxolyl, pyrrolidinyl, pyrrolidonyl, imidazolidinyl, pyrazolidinyl, tetrahydropyranyl, piperidinyl, piperazinyl, morpholinyl, thiomorpholinyl, dithianyl or trithianyl.

As used herein, when used alone or in combination with other groups, the term “aryl” means a monocyclic or fused polycyclic aryl having a conjugated it-electron system. For example, the term “C₆₋₁₀ aryl” used herein refers to an aryl having 6 to 10 carbon atoms. For example, the aryl may be phenyl, naphthyl, anthracenyl, phenanthrenyl, acenaphthyl, azulenyl, fluorenyl, indenyl, pyrenyl, etc.

As used herein, when used alone or in combination with other groups, the term “heteroaryl” means a monocyclic or fused polycyclic aromatic group having a conjugated it-electron system, the ring atoms of which are composed of carbon atoms and at least one heteroatom selected from N, O and S. The heteroaryl group may be attached to the rest of the molecule through any ring atom, provided the valence requirements are met. For example, the term “5-10 membered heteroaryl” used herein refers to a heteroaryl having 5 to 10 ring atoms. For example, the heteroaryl may be thienyl, furyl, pyrrolyl, oxazolyl, thiazolyl, imidazolyl, pyrazolyl, isoxazolyl, isothiazolyl, oxadiazolyl, triazolyl, thiadiazole, pyridyl, pyridazinyl, pyrimidinyl, pyrazinyl, triazinyl and benzo derivatives thereof, pyrrolopyridyl, pyrrolopyrazinyl, pyrazolopyridyl, imidazopyridyl, pyrrolopyrimidinyl, pyrazolopyrimidinyl, purinyl, etc.

As used herein, when used alone or in combination with other groups, the term “alkenyl” means a straight or branched chain aliphatic alkyl having one or more carbon-carbon double bonds. For example, the term “C₂₋₆ alkenyl” as used herein refers to an alkenyl having 2-6 carbon atoms and one, two or three (preferably one) carbon-carbon double bonds (such as vinyl, 1-propenyl, 2-propenyl, 2-butenyl, 3-butenyl, 2-pentenyl, 3-pentenyl, 4-pentenyl, 2-hexenyl, 3-hexenyl, 4-hexenyl, 5-hexenyl, 2-methyl-2-propenyl, 4-methyl-3-pentenyl, etc.).

As used herein, when used alone or in combination with other groups, the term “alkynyl” means a straight or branched chain aliphatic alkyl having one or more carbon-carbon triple bonds. For example, the term “C₂₋₆ alkynyl” as used herein refers to an alkynyl having 2-6 carbon atoms and one, two or three (preferably one) carbon-carbon triple bonds (such as ethynyl, 1-propynyl, 2-propynyl, 2-butynyl, 3-butynyl, 2-pentynyl, 3-pentynyl, 4-pentynyl, 2-hexynyl, 3-hexynyl, 4-hexynyl, 5-hexynyl, etc.).

As used herein, when used alone or in combination with other groups, the term “halo” or “halogen” group means F, Cl, Br or I.

As used herein, when used alone or in combination with other groups, the term “hydroxy” means —OH.

As used herein, when used alone or in combination with other groups, the term “cyano” means —CN.

As used herein, when used alone or in combination with other groups, the term “nitro” means —NO₂.

As used herein, when used alone or in combination with other groups, the term “amino” means —NH₂.

As used herein, when used alone or in combination with other groups, the term “oxo” means ═O.

The term “each independently” or “independently” used herein means that at least two groups (or fragments) present in a structure with the same or similar value range may have the same or different meanings under specific circumstances. For example, substituent X and substituent Y are each independently hydrogen, halogen, hydroxyl, —CN, alkyl or aryl, when the substituent X is hydrogen, the substituent Y is either hydrogen, or halogen, hydroxyl, —CN, alkyl or aryl; similarly, when the substituent Y is hydrogen, the substituent X is either hydrogen, or halogen, hydroxyl, —CN, alkyl or aryl.

The term “substituted” and other variants thereof herein mean that one or more (eg., 1, 2, 3 or 4) atoms or radicals (for example, hydrogen atoms) on the indicated atom are replaced by other equivalents, provided that the normal valence of the specified atom or radical is not exceeded for the circumstances, and that a stable compound is formed. If an atom or radical is described as “optionally substituted by . . . ”, it should be understood as either substituted or unsubstituted. Unless otherwise stated, the attachment point of a substituent herein may be from any suitable position of the substituent. When a linkage in a substituent is shown as passing through a bond between two interconnected atoms in a ring system, it means that the substituent may be linked to any ring-forming atom in the ring system.

The term “a pharmaceutically acceptable salt” means a salt of a compound according to the present application that is substantially non-toxic to an organism. Pharmaceutically acceptable salts generally include (but are not limited to) salts formed by reacting a compound according to the present application with a pharmaceutically acceptable inorganic acid/organic acid/acidic amino acid or inorganic base/organic base/basic amino acid, such salts are also known as acid addition salts or base addition salts. For a review of suitable salts see, for example, J Jusiak, Soczewinski, et al., Remington's Pharmaceutical Sciences [M], Mack Publishing Company, 2005; and Stahl, Wermuth, Handbook of Pharmaceutical Salts: Properties, Selection, and Use [M], Wiley-VCH, 2002. Methods for preparing a pharmaceutically acceptable salt of a compound according to the application are known to those skilled in the art.

The term “a pharmaceutically acceptable ester” means an ester that is substantially non-toxic to an organism and hydrolyzed into a compound according to the present application or a salt thereof in the organism. Furthermore, the compound per se according to the present application may be an ester.

The term “isomer” means a compound that has the same molecular weight due to the same number and type of atoms, but differ in the arrangement or configuration of the atoms in space.

The term “stereoisomer” (also known as “chiral isomer”) means a stable isomer that has a vertical asymmetric plane due to at least one chiral factor (including chiral center, chiral axis, chiral surface, etc.), thereby rotating plane-polarized light. Due to the presence of asymmetric centers and other chemical structures which may give rise to stereoisomerism in a compound according to the present application, the present application also encompasses these stereoisomers and mixtures thereof. Due to the fact that a compound (or a pharmaceutically acceptable salt thereof) according to the present application may comprise asymmetric carbon atoms, it may be present as a mixture of single stereoisomers, racemes, enantiomers, and diastereomers. The term “enantiomers” means a pair of stereoisomers with non-overlapping mirror images between them. The terms “diastereoisomer” or “diastereomer” mean optical isomers that are not mirror images to each other. The term “racemic mixture” or “racemate” means a mixture containing equal parts of a single enantiomer (i.e., an equimolar mixture of the two R and S enantiomers). The term “non-racemic mixture” refers to a mixture containing unequal parts of the individual enantiomers. Unless otherwise indicated, all stereoisomeric forms of the compounds according to the application are within the scope of the application.

The term “tautomer” (also known as “tautomeric form”) means structural isomers having different energies that are interconvertible through a low energy barrier. If tautomerism is possible (e.g., in a solution), a chemical equilibrium of the tautomers may be achieved. For example, proton tautomers (also known as prototropic tautomers) include (but are not limited to) interconversions by migration of a proton such as keto-enol isomerization, imine-enamine isomerization, amide-iminoalcohol isomerization, nitroso-oxime isomerization, etc. Unless otherwise indicated, all tautomeric forms of the compounds according to the application are within the scope of the application.

The term “solvate” means a substance formed by combination of a compound according to the present application (or a pharmaceutically acceptable salt thereof) with at least one solvent molecule through non-covalent intermolecular forces. The compounds according to the present application may exist in the form of solvates comprising polar solvents as elements of the crystal lattice structure. The amount of polar solvent may be present in stoichiometric or non-stoichiometric ratios.

The term “isotopic label” means a derivative compound formed by replacing a specific atom in a compound according to the present application with its isotopic atom. Unless otherwise indicated, the compounds according to the present application comprise the various isotopes of H, C, N, O, F, P, S, and Cl, such as ²H(D), ³H(T), ¹³C, ¹⁴C, ¹³N, ¹⁵N, ¹⁷O, ¹⁸O, ¹⁸F, ³¹P, ³²P, ³⁴S, ³⁵S, ³⁶S, ³⁷Cl and ¹²⁵I. For example, ¹²C may be replaced by ¹³C or ¹⁴C; ¹H may be replaced by ²H (D, deuterium) or ³H (T, tritium); ¹⁶O may be replaced by ¹⁸O, etc.

Those skilled in the art will understand that, not all nitrogen-containing heterocycles are capable of forming N-oxides, since nitrogen requires an available lone pair of electrons to be oxidized to oxides. Those skilled in the art will recognize nitrogen-containing heterocycles capable of forming N-oxides. Those skilled in the art will also recognize that, tertiary amines are capable of forming N-oxides. Synthetic methods for the preparation of N-oxides of heterocycles and tertiary amines are well known to those skilled in the art, for example, peroxyacids (such as peracetic acid and m-chloroperbenzoic acid (mCPBA)), hydrogen peroxide, alkyl peroxides (such as t-butyl hydroperoxide), sodium perborate and dioxiranes (such as dimethyldioxirane) may be used to oxidize heterocycles and tertiary amines. The methods for preparing N-oxides have been extensively described and reviewed in the literatures, see for example: T. L. Gilchrist, Comprehensive Organic Synthesis, vol. 7, pp 748-750 (A. R. Katritzky and A. J. Boulton, Eds., Academic Press); and G. W. H. Cheeseman and E. S. G. Werstiuk, Advances in Heterocyclic Chemistry, vol. 22, pp 390-392 (A. R. Katritzky and A. J. Boulton, Eds., Academic Press).

The term “metabolite” means a derivative compound formed after metabolism of the compound according to the present application, for example, a derivative compound is generated by oxidation, reduction, hydrolysis, amidation, deamidation, esterification enzymatic hydrolysis and other reactions. For further information on metabolism, see Goodman and Gilman's: The Pharmacological Basis of Therapeutics [M], McGraw-Hill International Editions, 199. The present application encompasses all possible metabolite forms of the compounds according to the present application, i.e., substances formed in the body of the individual to which the compounds according to the present application are administered. Metabolites of the compounds can be identified by techniques known in the art, and their activity can be characterized by assays.

The term “prodrug” means a derivative compound capable of directly or indirectly providing a compound according to the application upon administration to an individual. Particularly, preferred derivative compounds or prodrugs are compounds that increase the bioavailability of the compounds of the application (e.g., better absorption into the blood), or facilitate delivery of the parent compound to the site of action (e.g., the lymphatic system), when they are administered to a subject. Unless otherwise indicated, all prodrug forms of the compounds according to the application are within the scope of the application, and various prodrug forms are known in the art, see for example T. Higuchi, V. Stella, Pro-drugs as Novel Drug Delivery Systems [J], American Chemical Society, Vol. 14, 1975. Furthermore, the application also encompasses compounds of the application which comprise protecting groups. During any process of the preparations of the compounds according to the application, it may be necessary and/or desirable to protect sensitive or reactive groups on any of the molecules of interest, thereby forming chemically protected forms of the compounds according to the application. This may be achieved by conventional protecting groups, for example, those described in T T. W. Greene, P. G. M. Wuts, Protective Groups in Organic Synthesis [M], John Wiley & Sons, 2006. These protecting groups may be removed at an appropriate subsequent stage by methods known in the art.

As used herein, the term “effective amount” means the amount of an active ingredient which will achieve a desired effect to some extent after administration, e.g., alleviation of one or more symptoms of the condition being treated, or prevention of the condition or symptoms thereof.

As used herein, “individual” includes a human or non-human animal. Exemplary human individuals comprise human individuals suffering from a disease (e.g., a disease described herein) (referred to as a patient) or normal individuals. “Non-human animals” in the present application comprise all vertebrates, e.g., non-mammals (e.g., birds, amphibians, reptiles) and mammals, such as non-human primates, livestock and/or domesticated animals (e.g., sheep, dogs, cats, cows, pigs, etc.).

The compounds according to the present application may be formulated in combination with suitable pharmaceutically acceptable auxiliary materials into solid, semi-solid, liquid or gaseous preparations, for example, tablets, pills, capsules, powders, granules, ointments, emulsions, suspensions, suppositories, injections, inhalants, gels, microspheres and aerosols, etc.

Typical routes for administration of a compound according to the present application or a pharmaceutically acceptable form thereof include, but are not limited to: oral, rectal, local, inhalation, parenteral, sublingual, vaginal, nasal, intraocular, peritoneal, intramuscular, subcutaneous, and intravenous administration.

In some embodiments, the formulation is in oral form. For oral administration, the formulation may be prepared by mixing the active compound with a pharmaceutically acceptable excipient well known in the art. These excipients enable the compounds according to the present application to be formulated into tablets, pills, lozenges, dragees, capsules, liquids, gels, slurries, suspensions and the like for oral administration to patients.

A solid oral formulation may be prepared by conventional methods of mixing, filling or tabletting, for example, by mixing the active compound with a solid excipient, optionally milling the resulting mixture, adding other suitable excipients if necessary, and processing the mixture into granules to obtain tablets or the core of the sugar coating. Suitable excipients include, but are not limited to: binders, diluents, disintegrants, lubricants, glidants or flavoring agents, etc.

The compounds according to the present application may also be administered in the form of sterile injections, including sterile injectable aqueous or oily suspensions, or sterile injectable aqueous or oily solutions. The carrier that can be used includes, but not limited to: water, Ringer's solution, and isotonic sodium chloride solution. Furthermore, sterilized non-volatile oils such as mono- or di-glycerides, may be employed as a solvent or suspending medium.

The compounds according to the present application may also be suitable for parenteral administration, for example, sterile solutions, suspensions or lyophilized products in appropriate unit dosage forms.

Dosage regimens may be adjusted to provide the optimum desired response. For example, when administered in the form of an injection, a single bolus injection, a bolus injection and/or a continuous infusion, etc. may be administered. For example, several divided doses may be administered over time, or a dose may be proportionally reduced or increased as indicated by the exigencies of the therapeutic situation. It should be noted that, dosage values may vary with the type and severity of a condition to be alleviated, and may comprise a single dose or multiple doses. In general, the dosage for treatment will vary, depending on considerations, such as the age, sex, and general health of a patient to be treated; the frequency of treatment and the nature of the desired effect; the degree of tissue damage; the duration of symptoms; and other variables that can be adjusted by individual physicians. It is further understood that for any given individual, the specific dosing regimen will be adjusted over time according to the needs of the individual, the composition to be administered, and the professional judgment of a person administering the composition or supervising the administration of the composition. The administration amount and administration regimen of the pharmaceutical composition may be easily determined by those of ordinary skill in the clinical field. For example, the compound according to the present application may be administered 4 times a day in four divided doses to once every 3 days, and the dosage may be, for example, 0.01-1000 mg each time. The desired dosage may be administered in one or more times to obtain the desired result. The compounds according to the application may also be provided in unit dosage form. As another example, in all the administration methods of the compounds represented by the general formula described herein, the daily dosage is 0.01-50 mg/kg body weight, preferably 0.03-30 mg/kg body weight, more preferably 0.05-20 mg/kg body weight, in a single dose or divided doses.

Chemotherapy drugs include, for example, alkylating agents (nimustine, carmustine, cyclophosphamide, temozolomide, etc.), antimetabolites (5-fluorouracil, gemcitabine, methotrexate, etc.), antitumor antibiotics (doxorubicin, epirubicin, mitomycin, etc.), plant anticancer drugs (paclitaxel, vinblastine, etoposide, irinotecan, etc.), antitumor hormones (tamoxifen, megestrol, etc.), cisplatin, etc.

Adverse reactions caused by chemotherapy drugs include, for example, intestinal adverse reactions, bone marrow suppression, decreased immunity, organ weakness, and inflammation. Intestinal adverse reactions include, for example, loss of appetite, nausea and vomiting, oral ulcers, abdominal pain, and diarrhea.

The compounds according to the present application may be used alone, or may be used in combination with other known drugs for alleviating adverse reactions caused by chemotherapy drugs.

The compounds according to the present application may be used before, simultaneously with, or after chemotherapy drugs.

### BRIEF DESCRIPTION OF THE DRAWINGS

FIG. 1: Experiment on protection of ISX on the injury of small intestinal organoids in mice caused by 5-fluorouracil—immunofluorescence staining assay.

FIG. 2: Experiment on protection of ISX on the injury of small intestinal organoids in mice caused by 5-fluorouracil—bright field and live cells/dead cells double staining assay.

FIG. 3: Statistical results of experiment on protection of ISX on the injury of small intestinal organoids in mice caused by 5-fluorouracil—bright field and live cells/dead cells double staining assay.

FIG. 4: Experiment on protection of ISX on the injury of small intestinal organoids in mice caused by etoposide—immunofluorescence staining assay.

FIG. 5: Experiment on protection of ISX on the injury of small intestinal organoids in mice caused by etoposide—Western blotting assay.

FIG. 6: Experiment on protection of ISX on the injury of small intestinal organoids in mice caused by etoposide—bright field and live cells/dead cells double staining assay.

FIG. 7: Statistical results of experiment on protection of ISX on the injury of small intestinal organoids in mice caused by etoposide—bright field and live cells/dead cells double staining assay.

FIG. 8: Experiment on protection of ISX on human colon organoids—bright field assay.

FIG. 9: Statistical analysis results of experiments on protection of ISX on human colon organoids.

FIG. 10: Experiment on protection of ISX on human colon organoids—live cells/dead cells double staining assay.

FIG. 11: Protective effect of ISX on the intestinal tract of mice—TUNEL experiment.

FIG. 12: Protective effect of ISX on the intestinal tract of mice—the statistical results of TUNEL experiment.

FIG. 13: Comparative experiments on the protective effects of PD332991, CHIR99021 and ISX on intestinal organoids.

### DETAILED DESCRIPTION

### Experimental Materials and Animals

Animals: 6-8 week-old healthy C₅₇BL/6 mice, with 20-23 g of body weight, were purchased from BIOCYTOGEN Pharmaceutical Technology Co., Ltd. and bred in the SPF Laboratory Animal Center of Tsinghua University.

### Materials and Reagents:

ISX (selleck s7914),

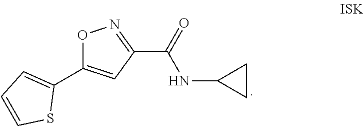 [SMILES: O=C(NC1CC1)c1cc(-c2cccs2)on1]

PD0332991, CHIR99021, 5-fluorouracil and etoposide (Selleck, S1579, S1263, S1209, S1225).

Human organ medium (STEMdiff intestinal organoid kit, STEMCELL, Cat #05140), and mouse organoid medium (intestiCult organoid growth medium, STEMCELL, 06005).

Cleaved caspase-3 antibody (CST, Cat #9661S); HRP-conjugated secondary antibody (Thermo Fisher, Cat. Nos. 31430 and 31460, respectively); β-tubulin antibody (SUNGENE BIOTECH Ltd., Cat #KM9003T); fluorescent secondary antibody Goat anti-Rabbit IgG (H+L) Cross-Adsorbed Secondary Antibody, Alexa Fluor 568 (Invitrogen, Cat #A-11011).

Live cells/dead cells double staining kit (Calcein-AM/PI) (BJBALB, Cat #HR0444), one-step TUNEL cell apoptosis detection kit (red fluorescence) (Beyotime, Cat #C1089).

PBS (M&C Gene Technology, Cat #CC008.1), Matrigel (Corning, Cat #254230), cell recovery solution (Corning, 354253), cell lysate (Beyotime, Cat #P0013), DAPI (Beyotime, C1002), Triton X-100 (sigma, Cat #9036-19-5), BSA (Albumin, M&C Gene Technology, LS000290), Tween-20 (sigma, 9005-64-5), immune fluorescence buffer (IF buffer): Triton X-100 (0.1%, diluted with PBS)+Tween-20 (0.05%, diluted with PBS), anti-fade agent (ProLong Gold anti-fade mountant, Invitrogen, Cat #P10144), proteinase K (Beyotime, Cat #ST532), ECL (Pierce ECL Plus Western Blotting Substrate, Cat #32132), Tween 80 (Merk, STS0204), CMC-Na (selleck, s6703).

Polyacrylamide (PAGE) gel: PAGE separating gel formulations and stacking gel formulations with different concentrations are shown in the following table:

Formulas of different concentrations of PAGE separating gel (lower gel)

| | The required volume for preparing 5 mL of gel | | | | | | | | | |
|---|---|---|---|---|---|---|---|---|---|---|
| Component | 6% | | 8% | | 10% | | 12% | | 15% | |
| 20M H₂O | 2.6 | mL | 2.3 | mL | 1.9 | mL | 1.6 | mL | 1.1 | mL |
| 30% AB | 1.0 | mL | 1.3 | mL | 1.7 | mL | 2 | mL | 2.5 | mL |
| Tris•HCl, | 1.3 | mL | 1.3 | mL | 1.3 | mL | 1.3 | mL | 1.3 | mL |
| pH 8.8 | | | | | | | | | | |
| 10% SDS | 50 | μL | 50 | μL | 50 | μL | 50 | μL | 50 | μL |
| 10% APS | 50 | μL | 50 | μL | 50 | μL | 50 | μL | 50 | μL |
| TEMED | 4 | μL | 3 | μL | 2 | μL | 2 | μL | 2 | μL |

The first 5 components were added in sequence according to the order in the above table. After confirming that the volume is correct and the gel plate has been set up, TEMED (Sigma, T7024) was added to the separating gel to mix well, then pouring the solution into the gap of the gel plate until the solution surface reached a position which is 1 cm away from the upper edge (generally, 0.75 mm thick glue needs to be perfused with 3.2-3.4 mL), adding 70% ethanol to flatten the liquid surface. The above gel plate was let to stand at room temperature for 30 minutes to 1 hour to fully solidify the gel, and it may be placed near a fluorescent tube to accelerate solidification. It should be noted that when the room temperature is low, white crystals may be formed in the 10% SDS solution. At this time, the SDS solution should be placed in a warm water bath at 37-60° C. to re-dissolve the SDS crystals and restore the solution to a uniform and clear state, then continuing to prepare PAGE gels.

Formulas of different volumes of PAGE stacking gel (top layer)

| Component | 2 mL | | 4 mL | | 6 mL | | 8 mL | | 10 mL | |
|---|---|---|---|---|---|---|---|---|---|---|
| 20M H2O | 1.4 | mL | 2.7 | mL | 4.1 | mL | 5.5 | mL | 6.8 | mL |
| 30% AB | 330 | μL | 670 | μL | 1 | mL | 1.3 | mL | 1.7 | mL |
| Tris•HCl, | 250 | μL | 500 | μL | 750 | μL | 1 | mL | 1.25 | mL |
| pH 6.8 | | | | | | | | | | |
| 10% SDS | 20 | μL | 40 | μL | 60 | μL | 80 | μL | 100 | μL |
| 10% APS | 20 | μL | 40 | μL | 60 | μL | 80 | μL | 100 | μL |
| TEMED | 2 | μL | 4 | μL | 6 | μL | 8 | μL | 10 | μL |

After the separating gel was solidified, the ethanol was suck up by vacuum pump. TEMED was added to the stacking gel solution to mix well, then immediately pouring the solution into the gap of the gel plate until it is filled up, without leaving any air bubbles. A gelling comb (a 10-hole or 15-hole comb was selected as needed) was inserted into the solution. The above gel plate was let to stand at room temperature for 20-30 minutes to completely solidify the gel solution. The gel is ready for electrophoresis immediately, or stored at 4° C. (to be used within 3 days). It is recommended to use a gel prepared freshly on the same day.

When preparing gradient gel, high-concentration PAGE separating gel solution was filled firstly, then immediately pouring low-concentration PAGE separating gel solution carefully and evenly to a until the solution surface reached a position which is 1 cm away from the top edge of the short glass plate, adding 70% ethanol to flatten the liquid surface, and the subsequent steps are the same as the preparation of a single-concentration PAGE.

70% ethanol: absolute ethanol was diluted with deionized water to 70% volume concentration, and it was used to flatten the liquid surface when preparing PAGE separating gel.

10% ammonium persulfate solution (10% APS): 5 g of ammonium persulfate powder (Sigma, A9164) was weighed to dissolve in 50 mL of deionized water, then dispensing into 1.5 mL tubes (300-500 μL per tube), and storing at −20° C.

10×SDS electrophoresis buffer (10×SDS Running Buffer): 151 g of Tris base, 470 g of glycine, and 250 g of SDS were used to prepare a 5 L solution with deionized water, then storing at room temperature.

1×SDS electrophoresis buffer (1 L): 100 mL of 10×SDS electrophoresis buffer was dissolved in 900 mL of deionized water.

10×Transmembrane buffer: 291.4 g of Tris base and 146.5 g of glycine were used to prepare a 5 L solution with deionized water, then storing at room temperature.

1χTransmembrane buffer (1 L): 100 mL of 10×Transmembrane buffer and 200 mL of methanol were dissolved in 700 mL of distilled water.

20×TBS (5 L): 800 g of NaCl, 20 g of KCl, and 300 g of Tris base were dissolved in deionized water, then adjusting the solution to pH 7.4 with concentrated hydrochloric acid, making up to a fixed volume, and storing at room temperature.

1×TBST (1 L): 50 mL of 20×TBS was added to a 1 L reagent bottle, when the volume was close to 1 L by adding deionized water, adding 500 μL of Tween-20 (AMRESCO), and making up to a fixed volume by adding deionized water.

Blocking solution (5% milk, TBST): 2.5 g of skimmed milk powder was weighed to dissolve in 45 mL of 1×TBST, then making up to a fixed volume of 50 mL. The solution should be prepared for immediate use.

Primary antibody diluent (5% BSA, TBST): 2.5 g of bovine serum albumin (BSA, M&C GENE TECHNOLOGY (BEIJING) LTD.) was weighed to dissolve in 45 mL of 1×TBST, then adding 150 μL of 10% sodium azide aqueous solution, and making up to a fixed 50 mL and storing at 4° C.

### Experimental Operations

### 1. Isolation of Crypts and Culture of Organoids

Mice with appropriate genotypes and genetic backgrounds were selected according to experimental requirements. To ensure the survival efficiency of organoids, generally, 6-8 week-old C₅₇BL/6 mice were selected as experimental materials. Before starting the experiment, the following operations were performed: pre-cooling the centrifuge to 4° C.; placing the pipette tip in the refrigerator to pre-cool; placing the multi-well cell culture plate into the cell culture incubator to pre-heat; and pre-cooling PBS and 5 mM EDTA (prepared with PBS) on ice.

(1) After the mice were sacrificed by cervical dislocation, the abdomen was placed upward on a 10 cm cell culture plate, and the abdomen was disinfected and moistened with 75% medical alcohol.

(2) The abdominal cavity was opened with scissors to take out the small intestine (note: removing the fat tissue on the outer wall of the intestine during the process of separating the small intestine), then cutting off the first half of the small intestine, and transferring it to a cell culture dish filled with pre-cooled PBS.

(3) The intestines were opened with surgical scissors, then clamping the small intestine tissues with forceps and rinsing it back and forth in PBS several times, and then discarding the PBS; rinsing intestines again with fresh PBS until the intestinal contents were cleaned.

(4) The small intestine tissues were cut into small pieces with a side length of about 4 mm, then transferring them to a cell culture dish added with pre-cooled 5 mM EDTA in advance, and allowing it to stand at 4° C. for 30 minutes.

(5) The small intestine tissues were transferred to a 50 ml centrifuge tube by using a tweezer, then adding 10 ml of pre-cooled PBS to shake vigorously up and down for 10 times (the villi of the small intestine will fall off at this point).

(6) The small intestine tissues were clamped back into a 50 ml centrifuge tube by using a tweezer, then adding 10 ml of pre-cooled PBS to shake vigorously up and down for 10 times, and pouring the liquid in the tube into a cell culture dish.

(7) After repeating the operation of the above step (6) once, the villi of the small intestinal tissues had basically fallen off, and the small intestinal tissues appeared as white translucent sheets.

(8) The above small intestinal tissues were clamped back into a 5 mM EDTA solution by using a tweezer, then standing at 4° C. for 30 minutes.

(9) The small intestine tissues were transferred to a 50 ml centrifuge tube, then adding 10 ml of pre-cooled PBS to shake up and down gently for 10 times, and pouring the liquid in the tube into a cell culture dish.

(10) The above small intestine tissues were clamped back into a 50 ml centrifuge tube by using a tweezer, then adding 10 ml of pre-cooled PBS to shake vigorously up and down for 10 times (a large number of crypts of the small intestine will fall off into the PBS). The PBS containing crypts was allowed to pass through a 70 micron sieve, then collecting it in a new 50 mL centrifuge tube (operating on ice).

(11) Repeating the operation of above step (10) 2 times.

(12) The collected crypts were centrifuged at 300×g for 2 minutes, then discarding the supernatant and placing the precipitate on ice, adding 1 ml of pre-cooled PBS to resuspend the crypts, and counting.

(13) An appropriate amount of crypt suspension was taken to dilute it to 100 crypts per microliter, drawing an equal volume of Matrigel to mix with the diluted crypt suspension evenly (operating on ice to prevent Matrigel from solidifying).

(14) the mixed Matrigel solution was dropped into a preheated multi-well cell culture plate, then flattening it as much as possible (but avoid touching the edge of the well), and adding 20 microliters of Matrigel to each well of a 24-well plate.

(15) The cell culture plate was put into a 37° C. incubator to stand for 10-15 minutes. Meanwhile, a small intestinal organoid medium should be pre-warmed.

(16) After Matrigel was fully solidified, the above preheated medium was added into the multi-well cell culture plate along the well wall, adding 500 microliters of the medium to each well of the 24-well plate, and adding the same volume of PBS to the remaining wells to prevent liquid evaporates unevenly.

(17) The above culture plate was transferred to a 37° C. cell culture incubator containing 5% carbon dioxide for culture, then observing the growth of the organoids the next day, and replacing the medium every other day.

### 2. Passage of Organoids

(1) The Matrigel wrapped with organoids was scraped off by using a 1 ml tip (cutting a small opening with scissors), then transferring it to a 15 ml centrifuge tube, and blowing it vigorously by replacing with a normal 1 ml tip until the organoids were dispersed.

(2) The above tube was centrifuged at 300×g for 1 minute, then discarding the supernatant, adding 10 ml of PBS to wash once, and then centrifuging at 200×g for 2 minutes, and discarding the supernatant.

(3) The precipitated organoid fragments were re-suspended with an appropriate amount of PBS to place them on ice, then mixing an appropriate volume of the suspension with an equal volume of Matrigel completely.

(4) The Matrigel solution was dropped into a preheated multi-well cell culture plate, flattening it as much as possible, then placing it into a cell culture incubator, and adding the medium after the Matrigel was fully solidified. The passage ratio is generally 1:4 to 1:6.

### 3. Immunofluorescence of Organoids

(1) Matrigel-coated organoids were carefully transferred to a 1.5 mL centrifuge tube by using a 1 mL pipette tip, then centrifuging at 500×g for 1 minute, and discarding the supernatant.

(2) Cell recovery solution was added to the above tube, then standing on ice for 40 minutes.

(3) The cell recovery solution was carefully removed, then washing once with PBS, adding 4% paraformaldehyde, and fixing overnight at 4° C.

(4) The paraformaldehyde was carefully removed, then washing twice with PBS (the organoids were placed on ice to deposit naturally).

(5) The PBS was removed, then adding permeation solution (Triton X-100, 1%, diluted with PBS), and permeating at room temperature for 20 minutes.

(6) The permeation solution was removed, then adding blocking solution (Triton X-100 (0.1%, diluted with PBS)+BSA (3%, diluted with PBS)), and blocking at room temperature for 1 hour.

(7) The blocking solution was removed, then adding the primary antibody diluted with blocking solution (1:1000), and standing overnight at 4° C.

(8) The primary antibody was carefully transferred into a centrifuge tube (recyclable).

(9) Immunofluorescence buffer (IF buffer) was added to the tube containing organoids, then flicking to mix well, washing at room temperature at 500 rpm for 5 minutes, standing for 2 minutes until the organoids deposited to the bottom of the tube, carefully removing the liquid. The above operations were repeated 3 times, trying to remove completely at the last time.

(10) A corresponding fluorescent secondary antibody was diluted with blocking solution (1:500), then adding it to the centrifuge tube containing the organoids, and incubating at room temperature for 45 minutes in the dark.

(11) The secondary antibody was carefully removed, then adding DAPI (1 μg/mL) diluted with immunofluorescence buffer to stain the nucleus for 10 minutes.

(12) The tube was washed 3 times with immunofluorescence buffer (the operations are the same as the above Step (9)).

(13) A spacer was pasted on a glass slide, then transferring the organoids to the wells of the spacer by using a 200 μL pipette tip with a small opening, and removing the remaining liquid completely.

(14) 12 μL of antifade reagent was added to the wells of the spacer.

(15) A cover glass was used to cover the spacer, then sealing it around with nail polish.

(16) The above glass slide was ventilated at room temperature for 1-2 hours in the dark. After the nail polish was fully solidified, observing and taking pictures, or storing at 4° C.; in the dark.

### 4. Calcein-AM/PI Live Cells/Dead Cells Double Staining Assay

Calcein-AM can permeate the cell membrane, and the esterase in living cells can remove its AM group to produce calcein and emit fluorescence. PI (propidium iodide) cannot pass through the intact cell membrane, and only after the cell membrane is damaged and the permeability changes, PI can enter the cell to combine with DNA and emit fluorescence. Therefore, Calcein-AM/PI can be a label for distinguishing the live or dead cells.

(1) Calcein-AM was dissolved in DMSO to prepare a 10 mM stock solution, which was stored in the dark at −20° C. after aliquoting. PI was dissolved with PBS to prepare a 1 mM stock solution, which was either stored in the dark at −20° C. after aliquoting, or stored at 4° C. for a short period of time.

(2) The Calcein-AM stock solution was diluted with PBS (diluted 20 times). The diluted Calcein-AM and PI stock solutions were respectively added to the culture medium and mixed evenly (their final concentrations were 0.5 μM and 1 μM, respectively) to make a staining working solution, which was heated in a water bath at 37° C. in the dark for 5 minutes.

(3) The culture medium was replaced with the staining working solution, then incubating in a 37° C. incubator for 20 minutes.

(4) The staining condition was observed under a fluorescent microscope, then taking pictures.

### 5. Western Blotting Assay

(1) Electrophoresis: constant voltage mode with an initial voltage of 80 volts was used. After the sample entered into the separating gel, the voltage was increased (up to 160 volt) until electrophoresis was completed.

(2) Membrane Transfer: generally, the wet method is used for membrane transfer. A PVDF membrane was soaked with methanol, then washing it once with deionized water and membrane transfer buffer, carefully covering with the entire polyacrylamide gel, and then covering with a filter paper and excluding air bubbles, placing them into the transfer tank after tightening the clip, adding the transfer buffer (choosing an appropriate ratio of methanol according to the size of the detected protein), and transferring at a constant flow of 300 mA for 1-3 hours. Then, a blocking solution can be prepared (a 5% skimmed milk powder solution was prepared with TBST solution).

(3) Blocking: after the membrane transfer was completed, the PVDF membrane was placed into a box containing blocking solution, then incubating on a shaker at room temperature for 1 hour.

(4) Primary antibody incubation: after blocking, the blocking solution was washed off with TBST, then cutting the PVDF membrane, adding primary antibody diluted in an appropriate ratio (appropriate antibody dilution buffer was used for different antibodies), and incubating overnight on a shaker at 4° C.

(5) After the primary antibody incubation, the PVDF membrane was washed 3 times with TBST at room temperature on a shaker, 10 minutes for each time.

(6) A secondary antibody diluted in an appropriate ratio (generally, the secondary antibody was diluted with TBST, and the special antibody was diluted with blocking solution) was added, then incubating at room temperature for 1 hour on a shaker.

(7) After the secondary antibody incubation was completed, the PVDF membrane was washed 3 times with TBST at room temperature on a shaker, 5-10 minutes for each time.

(8) An appropriate amount of ECL was dropped on the PVDF membrane for chemiluminescence detection, then exposing it with X-ray film in a dark room, or taking pictures by using an imager.

### 6. TUNEL Assay:

(1) Paraffin sections of mouse small intestine tissue were dewaxed in xylene for 5-10 minutes, dewaxing again for another 5-10 minutes in fresh xylene.

(2) The above tissue sections were dewaxed in absolute ethanol for 5 minutes, then treating with 90% ethanol for 2 minutes, 70% ethanol for 2 minutes, and distilled water for 2 minutes.

(3) 20 μg/ml DNase-free proteinase K was added dropwise on the tissue sections to react at 20-37° C. for 15-30 minutes.

(4) The tissue sections were washed 3 times with PBS or HBSS. Note: Proteinase K must be completely washed off in this step, otherwise, it will seriously interfere with the subsequent labeling reaction.

(5) The tissue sections were Incubated in 3% hydrogen peroxide solution prepared with PBS for 20 minutes at room temperature to inactivate endogenous peroxidase in the tissue sections, then washing with PBS or HBSS for 3 times.

(6) TUNEL detection solution was prepared by mixing 5 μl of TdT enzyme with 45 μl of fluorescent labeling solution.

(7) The tissue sections were washed twice with PBS or HBSS.

(8) 50 μl of TUNEL detection solution was added to the tissue samples, then incubating at 37° C. in the dark for 60 minutes.

(9) The tissue sections were washed 3 times with PBS or HBSS.

(10) After mounting, the tissue sections were observed under a fluorescence microscope, then taking pictures.

The compounds used in the Examples of the present application (such as ISX, 5-fluorouracil, etoposide, PD332991, CHIR99021) were used in the form of solutions dissolved in DMSO, and the concentrations of the compounds in the Examples were their final concentrations after adding them into the organoid medium.

### Example 1: Experiment on Protection of ISX on the Injury of Small Intestinal Organoids in Mice Caused by 5-FU—Immunofluorescence Staining Assay

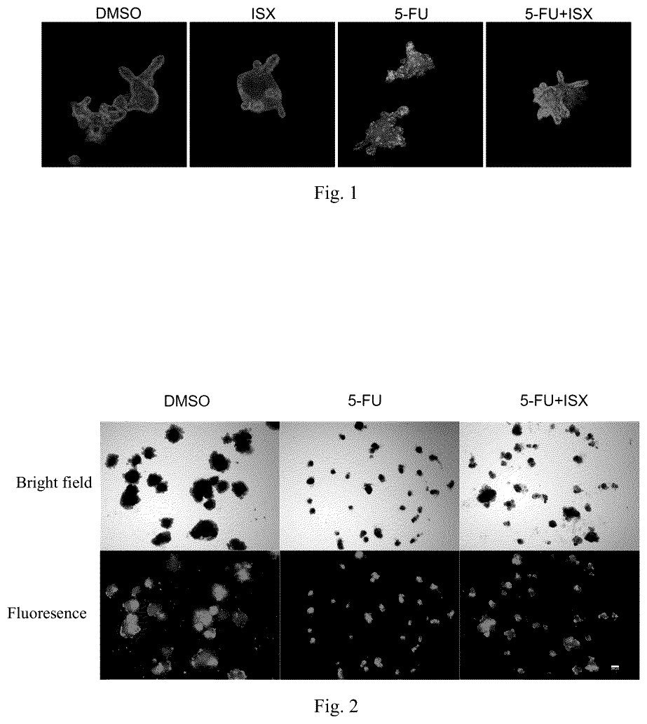

The small intestine tissues of 6-8 week-old mice were taken to perform organoid culture, after the culture was stable, performing subculture to continue the culture for 3-4 days, then adding the compound shown in FIG. 1 to the medium. The culture was dividing into 4 groups: a control group of adding DMSO alone (DMSO group); a group of adding the chemotherapeutic drug 5-fluorouracil (5-FU, 50 uM) alone (5-FU group); a group of adding ISX (25 uM) alone (ISX group); and a group of adding 5-FU (50 uM) and ISX (25 uM) simultaneously (5-FU+ISX group). The organoids were fixed after 4 hours of treatment, and the immunofluorescence staining assay was performed with the primary antibody against cleaved caspase-3, and the results were observed and recorded.

As shown in FIG. 1, the DAPI signal represents the nucleus, and the cleaved caspase-3 (cleaved caspase-3) signal represents the dead cells. It can be seen that, the dead cells in the 5-FU group are significantly higher than those in the DMSO group. Compared with the 5-FU group, the dead cells in the 5-FU+ISX group are significantly reduced, and the ISX group does not have a significant impact on the signal of the cleaved caspase-3. The above experiments show that ISX can alleviate the injury of the intestinal tissue caused by chemotherapy drug 5-fluorouracil.

### Example 2: Experiment on Protection of ISX on the Injury of Small Intestinal Organoids in Mice Caused by 5-FU—Bright Field and Live Cells/Dead Cells Double Staining Assay

The small intestine tissues of 6-8 week-old mice were taken to perform organoid culture, after the culture was stable, performing subculture to continue the culture for 2-3 days, then adding the compound shown in FIG. 2 to the medium. The culture was dividing into 3 groups: a control group of adding DMSO alone (DMSO group); a group of adding the chemotherapeutic drug 5-FU (50 uM) alone (5-FU group); and a group of adding 5-FU (50 uM) and ISX (25 uM) simultaneously (5-FU+ISX group). After 8 hours of treatment, all compound treatments were terminated, and the organoids were washed 3 times with PBS, then culturing in fresh medium. After 4 days of continuous culture, live cells/dead cells double staining assay was performed, and the organoids were observed under a microscope, then taking pictures and recording.

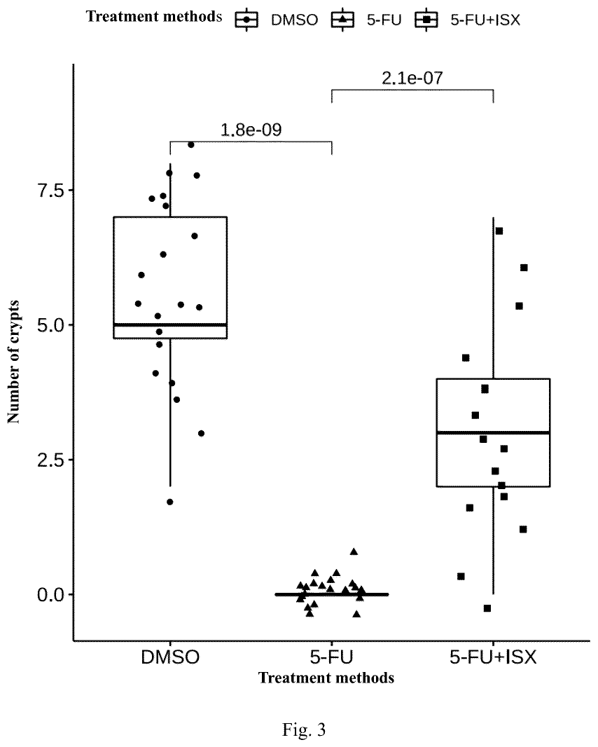

As shown in FIG. 2, it can be observed in the bright field image that, the biomass of the 5-FU group is significantly reduced as compared with that of the DMSO group, while the biomass of the 5-FU+ISX group is significantly increased as compared with that of the 5-FU group. The signal generated by PI in the fluorescence image represents dead cells, and the signal generated by Calcein-AM represents living cells. It can be observed that, the number of living cells in the 5-FU+ISX group is significantly more than that in the 5-FU group. Statistical analysis was performed on the number of surviving crypt structures in each organoid, as shown in FIG. 3, there is a significant difference between the 5-FU+ISX group and the 5-FU group (p<0.01). The above experiments show that ISX can alleviate the injury of small intestinal organoids in mice caused by chemotherapy drug 5-fluorouracil.

### Example 3: Experiment on Protection of ISX on the Injury of Small Intestinal Organoids in Mice Caused by Etoposide—Immunofluorescence Staining Assay

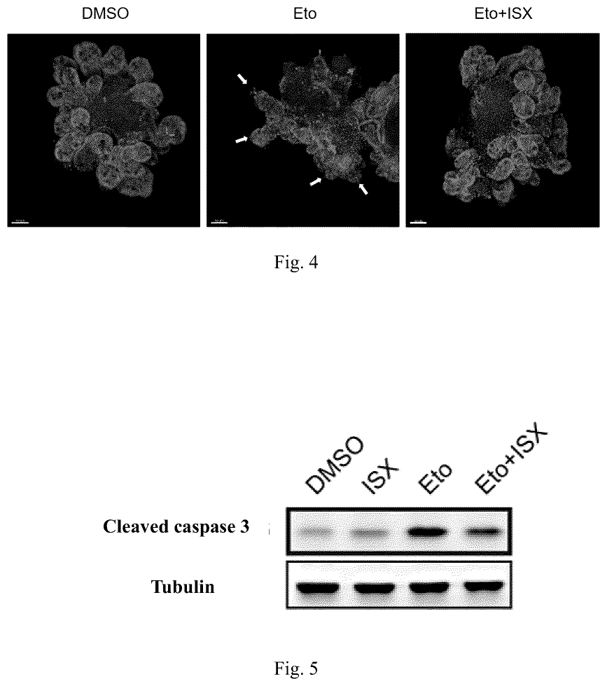

The small intestine tissues of 6-8 week-old mice were taken to perform organoid culture, after the culture was stable, performing subculture to continue the culture for 3-4 days, then adding the compound shown in FIG. 4 to the medium. The culture was dividing into 3 groups: a control group of adding DMSO alone (DMSO group); a group of adding chemotherapy drug etoposide (Eto, 50 uM) alone (Eto group); and a group of adding etoposide (Eto, 50 uM) and ISX (25 uM) simultaneously (Eto+ISX group). After 4 hours of treatment, the organoids were fixed, and the immunofluorescent staining assay was performed, and the organoids were observed under a microscope, then taking pictures and recording.

As shown in FIG. 4, the DAPI signal represents the nucleus, and the cleaved caspase-3 signal represents the apoptotic cell. It can be seen that, ISX can significantly reduce the cell death of small intestinal organoids in mice caused by etoposide.

### Example 4: Experiment on Protection of ISX on the Injury of Small Intestinal Organoids in Mice Caused by Etoposide-Western Blotting Assay

The small intestine tissues of 6-8 week-old mice were taken to perform organoid culture, after the culture was stable, performing subculture to continue the culture for 2-3 days, then adding the compound shown in FIG. 5 to the medium. The culture was dividing into 4 groups: a control group of adding DMSO alone (DMSO group); a group of adding ISX (25 uM) alone (ISX group); a group of adding chemotherapy drug etoposide (Eto, 50 uM) alone (Eto group); and a group of adding etoposide (Eto, 50 uM) and ISX (25 uM) simultaneously (Eto+ISX group). After 4 hours of treatment, 4 groups of samples were collected for Western blotting assay.

As shown in FIG. 5, ISX can significantly reduce the cell death of small intestinal organoids in mice caused by etoposide.

### Example 5: Experiment on Protection of ISX on the Injury of Small Intestinal Organoids in Mice Caused by Etoposide—Bright Field and Live Cells/Dead Cells Double Staining Assay

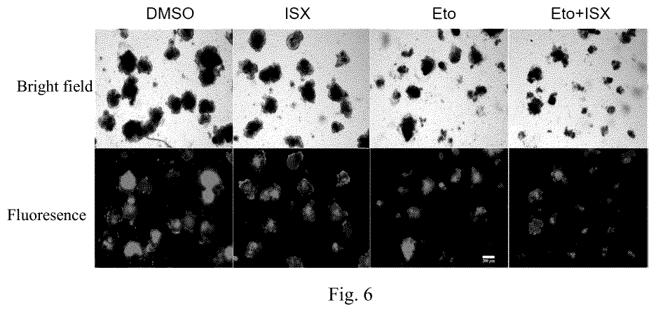

The small intestine tissues of 6-8 week-old mice were taken to perform organoid culture, after the culture was stable, performing subculture to continue the culture for 2-3 days, then adding the compound shown in FIG. 6 to the medium. The culture was dividing into 4 groups: a control group of adding DMSO alone (DMSO group); a group of adding ISX (25 uM) alone (ISX group); a group of adding chemotherapy drug etoposide (Eto, 50 uM) alone (Eto group); and a group of adding etoposide (Eto, 50 uM) and ISX (25 uM) simultaneously (Eto+ISX group). After 8 hours of treatment, all compound treatments were terminated, and the organoids were washed 3 times with PBS, then culturing in fresh medium. After 4 days of continuous culture, live cells/dead cells double staining assay was performed, and the organoids were observed under a microscope, then taking pictures and recording.

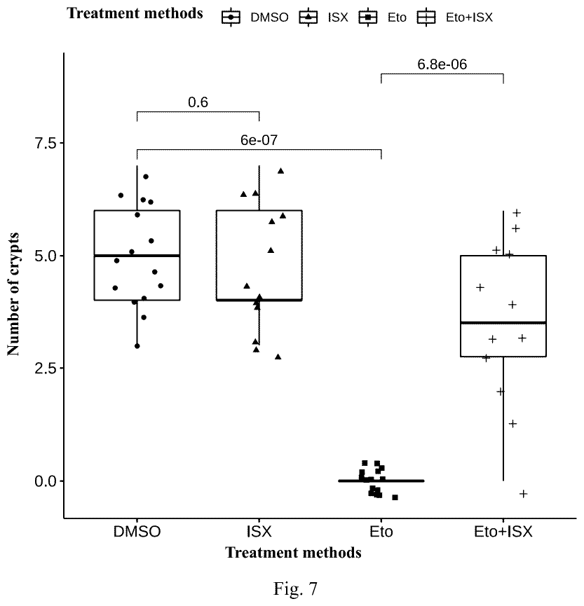

As shown in FIG. 6, it can be observed in the bright field images that, the biomass of the Eto group is significantly reduced as compared with that of the DMSO group, while the biomass of the Eto+ISX group is significantly increased as compared with that of the Eto group. The signal generated by PI in the fluorescence image represents dead cells, and the signal generated by Calcein-AM represents living cells. It can be observed that, the number of living cells in the Eto+ISX group is significantly more than that in the Eto group. Statistical analysis was performed on the number of surviving crypt structures in each organoid, as shown in FIG. 7, there is a significant difference between the Eto+ISX group and the Eto group (p<0.01). The above experiments showed that ISX can significantly reduce the cell death of small intestinal organoids in mice caused by etoposide.

### Example 6: Experiment on Protection of ISX on Human Colon Organoids—Bright Field and Live Cells/Dead Cells Double Staining Assay

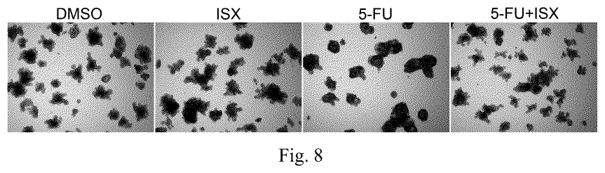

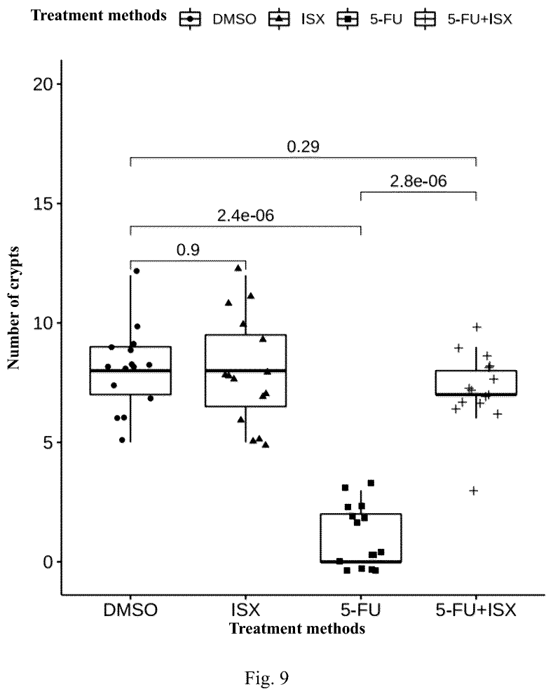

Colon tissues were isolated from normal human tissue to perform organoid culture, after the culture was stable, performing subculture to continue the culture for 3-5 days. The formation of crypt structure could be observed, then adding the compound shown in FIG. 8 to the medium. The culture was dividing into 4 groups: a control group of adding DMSO alone (DMSO group); a group of adding chemotherapy drug 5-fluorouracil (5-FU, 20 uM) alone (5-FU group); a group of adding ISX (10 uM) alone (ISX group); and a group of adding 5-FU (50 uM) and ISX (25 uM) simultaneously (5-FU+ISX group). After 48 hours of treatment, the organoids were observed under a microscope, then taking pictures to record the morphology of organoids in different treatment groups, and performing statistical analysis on the number of crypts in each organoid between different treatment groups (FIG. 8, FIG. 9).

As shown in FIGS. 8-9, the organoid crypt structures in the DMSO group are intact, while the number of crypt structures in the 5-FU group is significantly reduced. There is significant difference between the DMSO group and the 5-FU group (p<0.01). There is no significant difference between the 5-FU+ISX group and the DMSO group, and there is no significant difference between the 5-FU+ISX group and the DMSO group (p=0.29). There is no significant difference between ISX group and DMSO group (p=0.9). Compared with the 5-FU group, the number of crypts in the 5-FU+ISX group is significantly increased, and there is a significant difference between the 5-FU+ISX group and the 5-FU group.

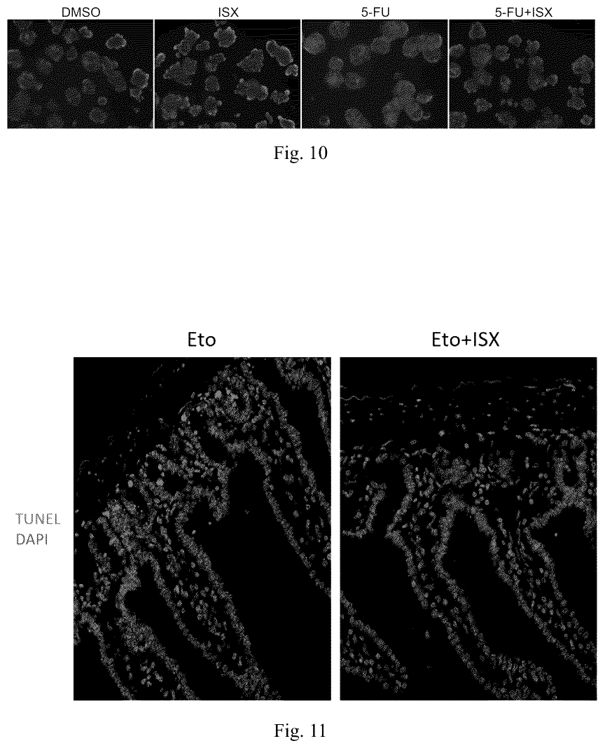

Continuing the culture until day 5, live cells/dead cells double staining assay was performed on all treatment groups, and the results were observed under a fluorescent microscope, then taking pictures to record. The signal generated by PI in the fluorescence image represents dead cells, and the signal generated by Calcein-AM represents living cells. As shown in FIG. 10, the dead cells in the 5-FU group are increased significantly as compared with the DMSO group, while there was no significant difference between ISX group and DMSO group. The above experiments show that ISX can alleviate the injury of human colon organoids caused by chemotherapy drug 5-fluorouracil.

### Example 7: Protective Effect of ISX on the Intestinal Tract of Mice—TUNEL Assay

6-8 week-old wild-type B6 mice were randomly divided into two groups. One group was intraperitoneally injected with chemotherapeutic drug etoposide (20 mg/kg), and then orally administered a cosolvent (5% DMSO+0.5% Tween 80+0.5% CMC-Na) (5 mL/kg); another group was intraperitoneally injected with chemotherapeutic drug etoposide (20 mg/kg), and then orally administered ISX dissolved in a cosolvent (20 mg/kg). The mice were sacrificed 3 hours later, and the intestinal tissues were fixed, followed by TUNEL staining assay.

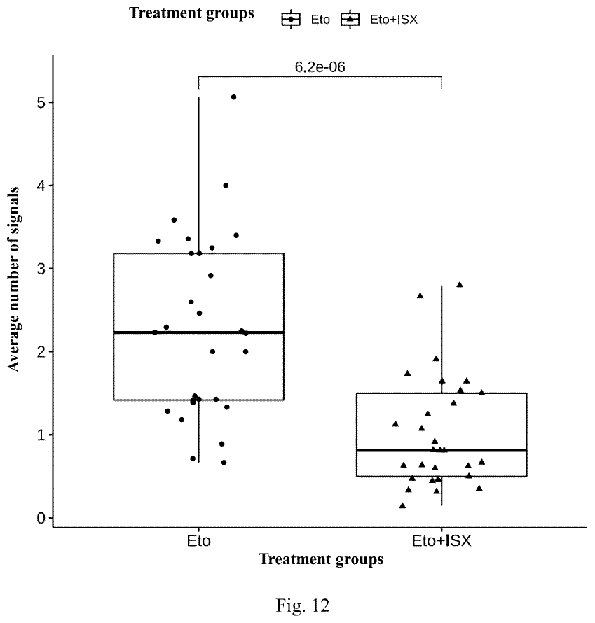

As shown in FIG. 11, the TUNEL signal of the orally administered ISX group is significantly lower than that of the orally administered cosolvent group. Statistical analysis was performed on the number of TUNEL signals in each crypt, and there is a significant difference between the two groups (p<0.01) (FIG. 12). The above experiments show that ISX can alleviate the injury of intestinal tract in mice caused by the chemotherapy drug etoposide.

### Example 8: Comparative Experiment on the Protective Effects of PD332991, CHIR99021 and ISX on Intestinal Organoids

PD0332991 is a CDK4/6 inhibitor, which has been reported to reduce the killing effect of chemotherapy drugs on normal cells. CHIR99021 is an inhibitor of GSK3β, which has been reported to alleviate the intestinal injury caused by chemotherapy drugs. In this example, PD0332991 and CHIR99021 were used as reference compounds to evaluate the protective effect of the compounds of the present application on intestinal organoids.

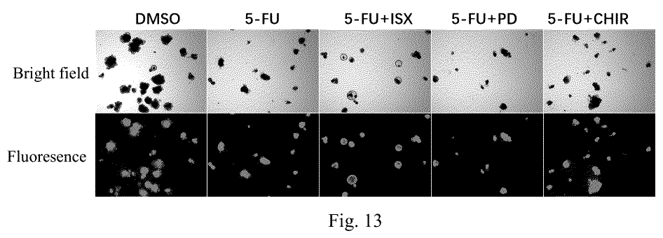

The small intestine tissues of 6-8 week-old mice were taken to perform organoid culture, after the culture was stable, performing subculture to continue the culture for 2-3 days, then adding the compound shown in FIG. 13 to the medium. The culture was dividing into 5 groups: a control group of adding DMSO alone (DMSO group); a group of adding 5-FU (50 uM) alone (5-FU group); a group of adding 5-FU (25 uM) and ISX (25 uM) simultaneously (5-FU+ISX group); a group of adding 5-FU (25 uM) and PD0332991 (100 nM) simultaneously (5-FU+PD group); a group of adding 5-FU (25 uM) and CHIR99021 (10 uM) simultaneously (5-FU+CHIR group). After treatment, all compound treatments were terminated, and the organoids were washed 3 times with PBS, then culturing in fresh medium. After 4 days of continuous culture, live cells/dead cells double staining assay were performed, and the organoids were observed under a microscope, then taking pictures and recording.

As shown in FIG. 13, ISX can significantly reduce the cell death of small intestinal organoids in mice caused by 5-FU treatment, while PD332991 and CHIR99021 cannot protect small intestinal organoids.

1. Use of a compound represented by formula (1) or a pharmaceutically acceptable form thereof in the preparation of a medicament for alleviating adverse reactions caused by chemotherapy drugs, the structure of the compound represented by formula (1) is as follows: 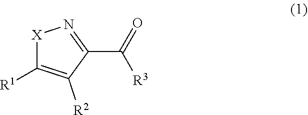
wherein:
R¹ is selected from C₁₋₆ alkyl, C₃₋₆ cycloalkyl, 3-8 membered heterocycloalkyl, C₆₋₁₀ aryl or 5-10 membered heteroaryl, and R¹ is optionally substituted by one or more R^{a};
the R^{a} is selected from hydrogen, hydroxyl, halogen, nitro, cyano, C₆₋₁₀ aryl, 5-10 membered heteroaryl, C₁₋₆ alkyl, —OC₁₋₆ alkyl, C₂₋₆ alkenyl, —OC₂₋₆ alkenyl, C₂₋₆ alkynyl, —OC₂₋₆ alkynyl, C₃₋₆ cycloalkyl, —C₁₋₆ alkylene-C₃₋₆ cycloalkyl, —OC₃₋₆ cycloalkyl, —OC₁₋₆ alkylene-C₃₋₆ cycloalkyl, —C₁₋₆ alkylene-C₆₋₁₀ aryl, —OC₆₋₁₀ aryl, —OC₁₋₆ alkylene-C₆₋₁₀ aryl, CHO, —(CO)R^{b}, —O(CO)R^{b}, —O(CO)OR^{b}, —C₁₋₆ alkylene-OR^{b}, —OC₂₋₆ alkylene-OR^{b}, —C₁₋₆ alkylene-(CO)R^{b}, —OC₁₋₆ alkylene-(CO)R^{b}, —CO₂R^{b}, —C₁₋₆ alkylene-CO₂R^{b}, —OC₁₋₆ alkylene-CO₂R^{b}, —NR^{b}R^{c}, —C₁₋₆ alkylene-NR^{b}R^{c}, —OC₂₋₆ alkylene-NR^{b}R^{c}, —C₁₋₆ alkylene-(CO)NR^{b}R^{c}, —OC₁₋₆ alkylene-(CO)NR^{b}R^{c}, —NR^{b}(CO)R^{c}, —C₁₋₆ alkylene-NR^{b}(CO)R^{c}, —OC₂₋₆ alkylene-NR^{b}(CO)R^{c}, —W(CO)NR^{b}R^{c}, —C₁₋₆ alkylene-NR^{b}(CO)NR^{b}R^{c}, —SR^{b}, —C₁₋₆ alkylene-SR^{b}, —OC₂₋₆ alkylene-SR^{b}, —(SO)R^{b}, —C₁₋₆ alkylene-(SO)R^{b}, —OC₂₋₆ alkylene-(SO)R^{b}, —SO₂R^{b}, —C₁₋₆ alkylene-SO₂R^{b}, —OC₂₋₆ alkylene-SO₂R^{b}, —(SO₂)NR^{b}R^{c}, —C₁₋₆ alkylene-(SO₂)NR^{b}R^{c}, —OC₁₋₆ alkylene-(SO₂)NR^{b}R^{c}, —NR^{b}(SO₂)R^{c}, —C₁₋₆ alkylene-NR^{b}(SO₂)R^{c}, —OC₂₋₆ alkyl ene-NR^{b}(SO₂)R^{c}, —W(SO₂)NR^{b}R^{c}, —C₁₋₆ alkylene-NR^{b}(SO₂)NR^{b}R^{c}, —OC₂₋₆ alkylene-NR^{b}(SO₂)NR^{b}R^{c}, —(CO)NR^{b}R^{c}, —O(CO)NR^{b}R^{c}, —NR^{b}OR^{c}, —NR^{b}(CO)OR^{c}, —C₁₋₆ alkylene-NR^{b}(CO)OR^{c} or —OC₂₋₆ alkylene-NR^{b}(CO)OR^{c}, and wherein the aryl, heteroaryl, alkyl, alkenyl, alkynyl, cycloalkyl, alkylene are optionally substituted by one or more substituents selected from the group consisting of: halogen, hydroxyl, cyano, nitro, C₁₋₆ alkyl, —OC₁₋₆ alkyl and C₃₋₆ cycloalkyl;
R² is selected from hydrogen, hydroxyl, halogen, nitro, cyano, C₆₋₁₀ aryl, 5-10 membered heteroaryl, C₁₋₆ alkyl, —OC₁₋₆ alkyl, C₂₋₆ alkenyl, —OC₂₋₆ alkenyl, C₂₋₆ alkynyl, —OC₂₋₆ alkynyl, C₃₋₆ cycloalkyl, —C₁₋₆ alkylene-C₃₋₆ cycloalkyl, —OC₃₋₆ cycloalkyl, —OC₁₋₆ alkylene-C₃₋₆ cycloalkyl, —C₁₋₆ alkylene-C₆₋₁₀ aryl, —OC₆₋₁₀ aryl, —OC₁₋₆ alkylene-C₆₋₁₀ aryl, CHO, —(CO)R^{b}, —O(CO)R^{b}, —O(CO)OR^{b}, —C₁₋₆ alkylene-OR^{b}, —OC₂₋₆ alkylene-OR^{b}, —C₁₋₆ alkylene-(CO)R^{b}, —OC₁₋₆ alkylene-(CO)R^{b}, —CO₂R^{b}, —C₁₋₆ alkylene-CO₂R^{b}, —OC₁₋₆ alkylene-CO₂R^{b}, —NR^{b}R^{c}, —C₁₋₆ alkylene-NR^{b}R^{c}, —OC₂₋₆ alkylene-NR^{b}R^{c}, —C₁₋₆ alkylene-(CO)NR^{b}R^{c}, —OC₁₋₆ alkylene-(CO)NR^{b}R^{c}, —NR^{b}(CO)R^{c}, —C₁₋₆ alkylene-NR^{b}(CO)R^{c}, —OC₂₋₆ alkylene-NR^{b}(CO)R^{c}, —NR^{b}(CO)NR^{b}R^{c}, —C₁₋₆ alkylene-NR^{b}(CO)NR^{b}R^{c}, —SR^{b}, —C₁₋₆ alkylene-SR^{b}, —OC₂₋₆ alkylene-SR^{b}, —(SO)R^{b}, —C₁₋₆ alkylene(SO)R^{b}, —OC₂₋₆ alkylene-(SO)R^{b}, —SO₂R^{b}, —OC₂₋₆ alkylene-(SO)R^{b}, —SO₂R^{b}, —C₁₋₆ alkylene-SO₂R^{b}, —OC₂₋₆ alkylene-SO₂R^{b}, —(SO₂)NR^{b}R^{c}, —C₁₋₆ alkylene-(SO₂)NR^{b}R^{c}, —OC₂₋₆ alkylene-(SO₂)NR^{b}R^{c}, —NR^{b}(SO₂)R^{c}, —C₁₋₆ alkylene-NR^{b}(SO₂)R^{c}, —OC₂₋₆ alkylene-NR^{b}(SO₂)R^{c}, —NR^{b}(SO₂)NR^{b}R^{c}, —C₁₋₆ alkylene-NR^{b}(SO₂)NR^{b}R^{c}, —OC₂₋₆ alkylene-NR^{b}(SO₂)NR^{b}R^{c}, —(CO)NR^{b}R^{c}, —O(CO)NR^{b}R^{c}, —NR^{b}OR^{c}, —NR^{b}(CO)OR^{c}, —C₁₋₆ alkylene-NR^{b}(CO)OR^{c} or —OC₂₋₆ alkylene-NR^{b}(CO)OR^{c}, and wherein the aryl, heteroaryl, alkyl, alkenyl, alkynyl, cycloalkyl, alkylene are optionally substituted by one or more substituents selected from the group consisting of: halogen, hydroxyl, cyano, nitro, C₁₋₆ alkyl, —OC₁₋₆ alkyl and C₃₋₆ cycloalkyl;
R^{b} and R^{c} are independently selected from hydrogen, C₁₋₆ alkyl, —C₁₋₆ alkylene-C₆₋₁₀ aryl, C₁₋₆ alkylene-(5-10 membered heteroaryl), C₃₋₇ cycloalkyl or C₆₋₁₀ aryl;
R³ is selected from —OR⁷ or —NR⁷R⁸;
R⁷ and R⁸ are independently selected from hydrogen, hydroxyl, C₁₋₆ alkyl, —OC₁₋₆ alkyl, C₂₋₆ alkenyl, C₂₋₆ alkynyl, —C₁₋₆ alkylene-C₆₋₁₀ aryl, —C₁₋₆ alkylene-(5-10 membered heteroaryl), —C₁₋₆ alkylene-(3-8 membered heterocycloalkyl), C₃₋₇ cycloalkyl or C₆₋₁₀ aryl, and wherein the aryl, heteroaryl, alkyl, alkenyl, alkynyl, cycloalkyl, and alkylene are optionally substituted by one or more substituents selected from the group consisting of: halogen, hydroxyl, cyano, nitro, C₁₋₆ alkyl, —OC₁₋₆ alkyl and C₃₋₆ cycloalkyl;
alternatively, R⁷ and R⁸ form a 3-8 membered heterocycloalkyl or a 5-10 membered heteroaryl together with the N atom to which they are attached;
X is selected from O or NR^{d}, and R^{d} is selected from hydrogen, C₁₋₆ alkyl, —C₁₋₆ alkylene-C₆₋₁₀ aryl, —C₁₋₆ alkylene-(5-10 membered heteroaryl), C₃₋₇ cycloalkyl or C₆₋₁₀ aryl; and
the pharmaceutically acceptable form is selected from pharmaceutically acceptable salts, esters, stereoisomers, tautomers, solvates, N-oxides, isotopic labels, metabolites or prodrugs.

2. The use according to claim 1, wherein R¹ is selected from C₁₋₆ alkyl, C₆₋₁₀ aryl or 5-10 membered heteroaryl; preferably, R¹ is selected from pyridyl, methyl, phenyl, thienyl, benzothienyl, furyl, benzofuryl, pyrrolyl or thiazolyl; more preferably, R¹ is selected from

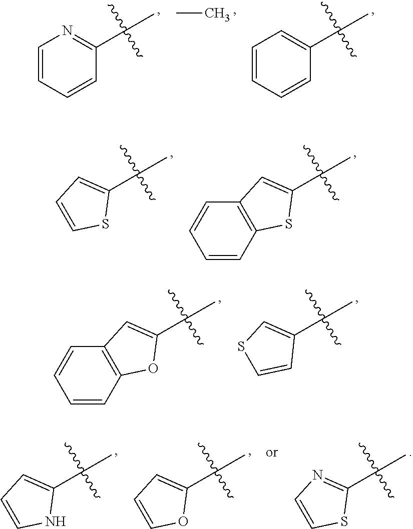

3. The use according to claim 1, wherein the compound represented by the formula (1) is a compound represented by the following formula (2): 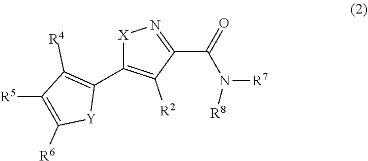
wherein:
R⁴, R⁵ and R⁶ are independently selected from hydrogen, hydroxyl, halogen, nitro, cyano, C₆₋₁₀ aryl, 5-10 membered heteroaryl, C₁₋₆ alkyl, —OC₁₋₆ alkyl, C₂₋₆ alkenyl, —OC₂₋₆ alkenyl, C₂₋₆ alkynyl, —OC₂₋₆ alkynyl, C₃₋₆ cycloalkyl, —C₁₋₆ alkylene-C₃₋₆ cycloalkyl, —OC₃₋₆ cycloalkyl, —OC₁₋₆ alkylene-C₃₋₆ cycloalkyl, —C₁₋₆ alkylene-C₆₋₁₀ aryl, —OC₆₋₁₀ aryl, —OC₁₋₆ alkylene-C₆₋₁₀ aryl, CHO, —(CO)R^{b}, —O(CO)R^{b}, —O(CO)OR^{b}, —C₁₋₆ alkylene-OR^{b}, —OC₂₋₆ alkylene-OR^{b}, —C₁₋₆ alkylene-(CO)R^{b}, —OC₁₋₆ alkylene-(CO)R^{b}, —CO₂R^{b}, —C₁₋₆ alkylene-CO₂R^{b} or —OC₁₋₆ alkylene-CO₂R^{b}, and wherein the aryl, heteroaryl, alkyl, alkenyl, alkynyl, cycloalkyl, and alkylene are optionally substituted by one or more substituents selected from the group consisting of: halogen, hydroxyl, cyano, nitro, C₁₋₆ alkyl, —OC₁₋₆ alkyl and C₃₋₆ cycloalkyl;
Y is selected from S, O or NH; and
X, R^{b}, R², R¹ and R⁸ are as defined in claim 1.

4. The use according to claim 3, wherein the compound represented by formula (2) is a compound represented by formula (3): 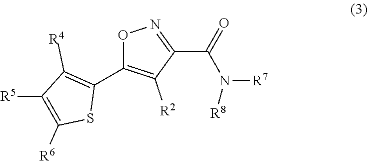
wherein R⁴, R⁵, R⁶, R², R⁷ and R⁸ are as defined in claim 3.

5. The use according to claim 4, wherein R⁴, R⁵ and R⁶ are independently selected from hydrogen, hydroxyl, halogen, nitro, cyano, C₆₋₁₀ aryl, 5-10 membered heteroaryl, C₁₋₆ alkyl, —OC₁₋₆ alkyl, C₂₋₆ alkenyl, —OC₂₋₆ alkenyl, C₂₋₆ alkynyl, —OC₂₋₆ alkynyl, C₃₋₆ cycloalkyl, —OC₃₋₆ cycloalkyl or —OC₆₋₁₀ aryl, and wherein the aryl, heteroaryl, alkyl, alkenyl, alkynyl, cycloalkyl are optionally substituted by one or more substituents selected from the group consisting of: halogen, hydroxyl, cyano, nitro and C₁₋₆ alkyl;
preferably, R⁴, R⁵ and R⁶ are independently selected from hydrogen, hydroxyl, halogen, nitro, cyano, C₆₋₁₀ aryl or C₁₋₆ alkyl; more preferably, R⁴, R⁵ and R⁶ are independently selected from hydrogen, hydroxyl, halogen, nitro, cyano, phenyl, methyl or n-butyl.

6. The use according to claim 1, wherein R² is selected from hydrogen, hydroxyl, halogen, nitro, cyano, C₆₋₁₀ aryl, 5-10 membered heteroaryl, C₁₋₆ alkyl, —OC₁₋₆ alkyl, C₂₋₆ alkenyl, —OC₂₋₆ alkenyl, C₂₋₆ alkynyl, —OC₂₋₆ alkynyl, C₃₋₆ cycloalkyl, —OC₃₋₆ cycloalkyl, —OC₆₋₁₀ aryl or CHO; preferably, R² is selected from hydrogen, hydroxyl, halogen, nitro, cyano or methyl.

7. The use according to claim 1, wherein R⁷ and R⁸ are independently selected from hydrogen, hydroxyl, C₁₋₆ alkyl, —OC₁₋₆ alkyl, —C₁₋₆ alkylene-(5-10 membered heteroaryl), —C₁₋₆ alkylene-(3-8 membered heterocycloalkyl), C₃₋₇ cycloalkyl or C₆₋₁₀ aryl, and wherein the aryl, heteroaryl, alkyl, cycloalkyl, and alkylene are optionally substituted by one or more substituents selected from the group consisting of: halogen, hydroxyl, cyano, nitro, and C₁₋₆ alkyl; alternatively, R⁷ and R⁸ form a 3-8 membered heterocycloalkyl together with the N atom to which they are attached;
preferably, R⁷ and R⁸ are independently selected from hydrogen, hydroxyl, cyclopropyl, cyclobutyl, cyclopentyl, cyclohexyl, tert-butyl, 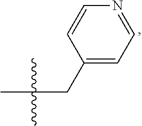 [SMILES: *Cc1ccncc1]
methyl n-butyl 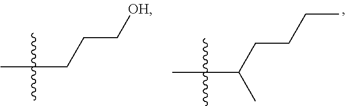
n-propyl, 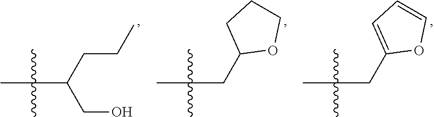
methoxy, ethoxy, or

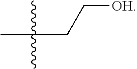 [SMILES: *CCO]

8. Use of a compound or a pharmaceutically acceptable form thereof in the preparation of a medicament for alleviating adverse reactions caused by chemotherapeutic drugs, wherein the compound is selected from the group consisting of: 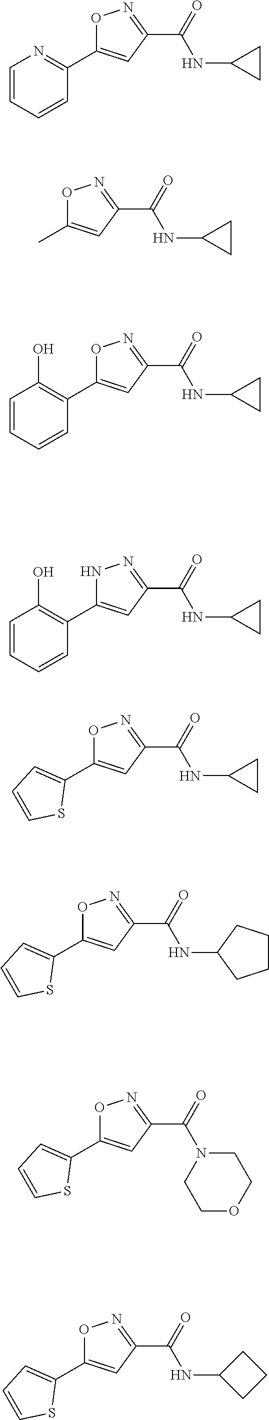 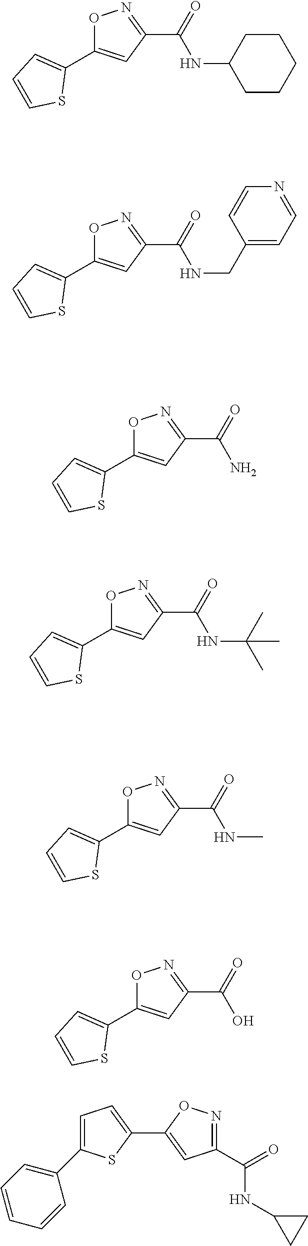 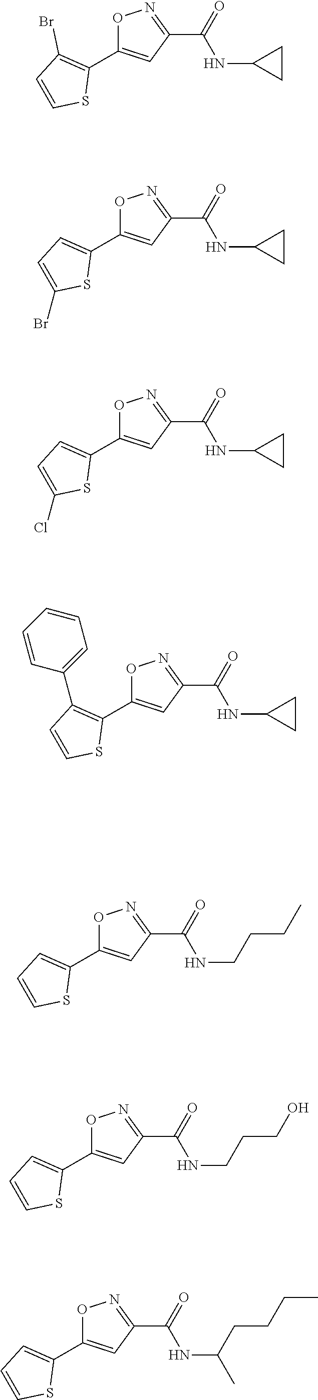 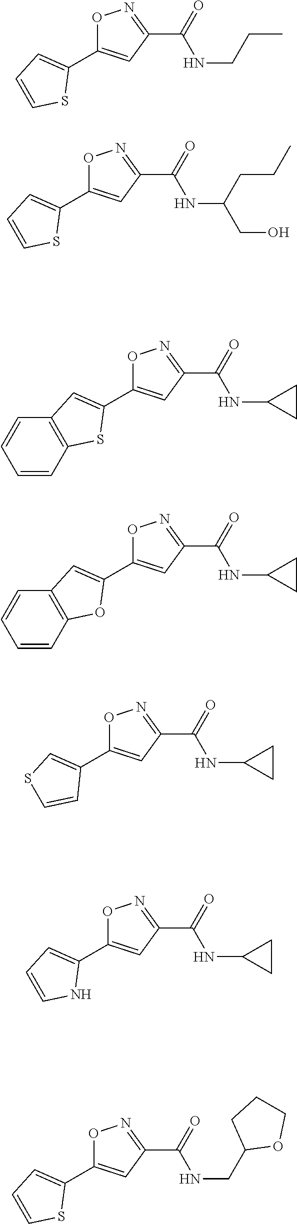 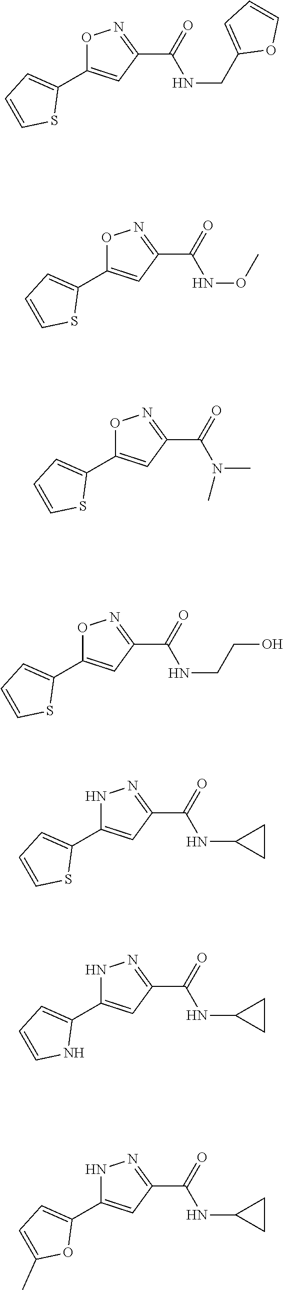 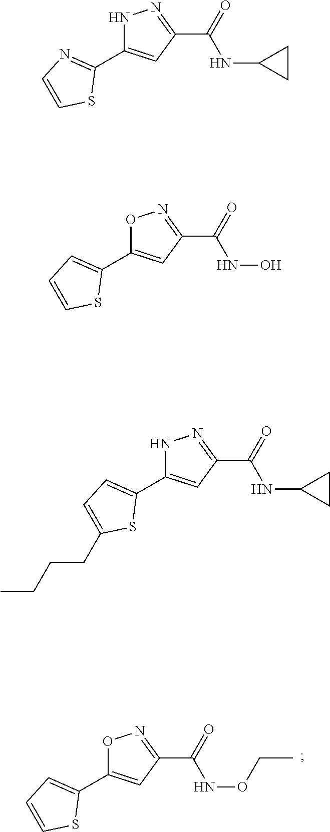 [SMILES: O=C(NC1CC1)c1cc(-c2nccs2)[nH]n1 ; O=C(NO)c1cc(-c2cccs2)on1 ; CCCCc1ccc(-c2cc(C(=O)NC3CC3)n[nH]2)s1 ; CCONC(=O)c1cc(-c2cccs2)on1]
and
wherein the pharmaceutically acceptable form is selected from pharmaceutically acceptable salts, esters, stereoisomers, tautomers, solvates, N-oxides, isotopic labels, metabolites or prodrugs.

9. The use according to claim 1, wherein the adverse reaction is an intestinal adverse reaction.
